(19) United States
(12) Patent Application Publication
(10) Pub. No.: US 2026/0130789 A1
(43) Pub. Date: May 14, 2026

(54) DEPLOYMENT DEVICE FOR AN INSERTABLE AND METHODS OF USE AND MANUFACTURE

(71) Applicant: Meditati Pty Ltd, Chatswood, NSW (AU)

(72) Inventor: Tonia Price, Chatswood, NSW (AU)

(21) Appl. No.: 19/118,564

(22) PCT Filed: Oct. 4, 2022
(86) PCT No.: PCT/AU2022/051183

§ 371 Date: Apr. 4, 2025

(51) Int. Cl. A61F 6/12; A61F 13/20; A61F 13/26; A61F 13/28; A61M 31/00

(52) U.S. Cl. CPC A61F 6/12; A61F 13/2091; A61F 13/266; A61F 13/28; A61M 31/007

(57) ABSTRACT

A delivery device for deploying an insertable into an orifice guided by a digit includes: an insertable orientation means having a first longitudinal axis and a base portion, the insertable orientation means adapted to load an insertable positioned in a deployment orientation, and a skirt member extending from adjacent the insertable orientation means and having a skirt length and comprising opposing digit contacting and orifice contacting surfaces, wherein the skirt member is adapted to be movable substantially parallel to the first longitudinal axis and along the skirt length, between: an insertable surrounding configuration, wherein an orifice contacting length of the orifice contacting surface substantially faces the insertable orientation means and any loaded insertable; and a digit surrounding configuration, wherein the orifice contacting length of the orifice contacting surface substantially faces away from the insertable orientation means and any loaded insertable.

### CROSS-REFERENCE TO RELATED APPLICATIONS

This application is a national phase entry under 35 U.S.C. § 371 of International Patent Application PCT/AU2022/051183, filed Oct. 4, 2022, designating the United States of America and published as International Patent Publication WO 2024/073791 A1 on Apr. 11, 2024.

### TECHNICAL FIELD

The present disclosure relates to devices for deploying an insertable into an orifice. Specifically, the present disclosure relates to devices and methods for deploying an insertable into an orifice guided by digit. The present disclosure also relates to methods of manufacturing such devices.

### BACKGROUND

The insertion of a variety of insertables into orifices, such as the vaginal orifice or anus, for treatment and/or diagnosis of a variety of conditions, and for a variety of other purposes, is known. For example, tampons are used by persons during their menstrual period to absorb menstrual fluid and the like. The use of tampons can be associated with significant risk of infection and disease. For example, urinary tract infections have been linked to a higher risk of developing urinary tract cancers (Japanese Cancer Association onlinelibrary.wiley.com/doi/full/10.1111/cas.12127). This can be precipitated by poor hygiene control during tampon insertion and/or removal.

There are essentially two modes of inserting tampons that are currently in use. The first mode is via direct digital insertion. The user inserts the tampon into the vagina with a finger(s). This can be unhygienic and invasive and, in some instances, an embarrassing procedure for a user, particularly when in public facilities. For the uninitiated, i.e., persons who have just started experiencing a menstruation, the procedure can be tricky and painful. The contact between the finger and the vaginal wall may potentially result in foreign micro-organisms being introduced into the vagina, together with an increased likelihood of the procedure soiling the inserter's fingers.

There can be lack of basic hygiene when items such as digitally insertable tampons are inserted or removed. Visible blood can remain on the fingers of a user that can be transferred to clothes, doors, taps and the like, whatever is touched post insertion and before washing, which is unhygienic and unpleasant for the next user.

The second current mode of inserting tampons is via an applicator, which is typically made from cardboard, plastic material or organic materials. Tampon applicators currently on the market are generally considered to provide for a more hygienic process than digital insertion. Furthermore, tampon applicators are sometimes considered to make insertion of the tampon more reliable and correct.

The problems with current tampon applicators include that they are difficult to handle (often involving multiple movable parts) and require relatively bulky packaging. Further, tampon applicators can cause pinching of the vaginal wall. Tampon applicators are invasive and have a significant environmental impact upon disposal. Blood soiled applicators are also a biological hazard. Given their typical length, tampon applicators are more conspicuous and more difficult to be transported and handled in a discreet manner. Furthermore, the tampon encased within the many tampon applicators cannot be inspected prior to insertion, which has been known to result in a user inserting an unsanitary tampon. Tampon applicators may also require some manual dexterity to operate, which can be particularly challenging for new and inexperienced users, and for people with disabilities.

Suppositories are recognized as accepted alternative formulations for pharmaceutical delivery. Rectal suppositories are used to administer of a variety of medications via the rectum. They are conveniently used in pediatric and geriatric populations. However, persons of all ages use rectal suppositories. Vaginal suppositories and pessaries are also used to administer vaginal contraceptives, pain relief and to treat various gynecological conditions. Pessaries come in different sizes and shapes, including, for example, doughnut pessaries, ring pessaries with supports, and Gellhorn pessaries. Pessaries may also be used to provide support to vaginal tissues displaced by pelvic organ prolapse. As with tampons, digital insertion is often used to insert a suppository or pessary and carries similar biological hazard risks, and some or all of the other difficulties identified above associated with digital insertion of tampons.

WO 2006/086849 discloses an insertable member for digital insertion into an orifice of a human or animal, which may be used to insert a tampon. The insertion member comprises an elongate insertion body to be received within the orifice and a sheath member that is detachably connected to at least a portion of the elongate insertion body. The sheath member is moveable relative to the elongate insertion body between a first configuration and a second configuration such that when the sheath member is in the second configuration, the sheath member is dimensioned to receive at least part of one or more digits of a user. The sheath member contains a tampon, suppository, tablet, or pessary and is hygienically sealed at the top end. However, this invention was found not to allow for a secure finger hold.

WO2005/058218 and WO2006/123132 disclose sanitary products for use by persons for the absorption of menstrual fluid comprising a plug (which is the word used in both applications to reference a tampon) and a pad joined by a sheath. In situ, when the plug is in the vagina cavity, the pad remains outside the vagina, but has an absorbent layer on an inward side proximal to the plug and a liquid impermeable layer on an outward side. The sheath can comprise a tube of absorbent material for drawing excess menstrual fluid from the plug to the pad and has a layer of liquid impermeable material on its inside surface. A string is provided that extends inside the sheath from the outward end of the plug to aid removal of the product.

In use of the invention disclosed in WO2005/058218 and WO2006/123132, a wearer can insert a finger into the sheath from the outward side of the pad to assist with insertion of the plug into the vaginal cavity. In that configuration, a proximal portion of the plug touches the tip of the user's finger, unless in an embodiment wherein the thin film surrounding the proximal portion of the plug is substantially interposed between the tip of the user's finger and the proximal portion of the plug. In those embodiments, the film surrounding the proximal portion of the plug is so thin that a user nevertheless feels as though she is touching the proximal end of the plug. However, the combination of plug and pad can be bulky and obtrusive to insert and place and also to remove and dispose of. Although both patent applications indicate that the pad can be moved away from the urethral opening so the pad is not soiled by urine during micturition, the practical reality is that the pad is likely to be exposed to urine irrespective.

WO2021/237269 A1 discloses delivery devices for deploying insertables into an orifice guided by digit. The delivery devices comprise an insertable orientation means adapted to orient the insertable in a deployment orientation and digit orientation means adapted to orient a portion of at least one digit in a navigation orientation, the insertable orientation means and the digit orientation means adjoined across a partition with a partition cross-section, and the deployment orientation and the navigation orientation are arranged for digit guided deployment of the insertable into the orifice. Also disclosed is a method of deploying an insertable into an orifice guided by digit, using the delivery device of the disclosed invention. The delivery device and method of the disclosed invention are said to be well suited to deploying tampons into the vaginal cavity. However, this invention was found to require a manufacturing approach that was intricate, bringing associated complexities for maintaining stringent quality control, and expensive, straining commercial viability.

Another significant issue for tampon applicator products is an inability to dispose of a used applicator by flushing down the toilet, because current applicators are not currently manufactured using dissolvable or flushable materials. Applicators that are not made from dissolvable material, but are nonetheless flushed down a toilet, can cause significant damage and blockage in sewerage system pipes.

WO 2018/102155 A1 to Kimberly-Clark Worldwide, Inc. discloses a flushable tampon applicator product that includes an outer tube for housing a tampon; an inner tube, at least a portion of which extends into the outer tube, wherein the outer tube includes an outer, body-contacting surface, wherein the inner tube is moveable relative to the outer tube and configured to expel a tampon from the outer tube, and wherein at least one of the outer tube and the inner tube comprises a thermoplastic composition including partially-hydrolyzed polyvinyl alcohol (PVOH), polyethylene glycol (PEG), a plasticizer, and a hydrophobic polymeric component. However, such barrel tampon applicators can be awkward and obtrusive to use, and difficult for young and inexperienced users, or people with disabilities, as it is difficult to determine as to whether placement is correct.

Another shortcoming of some prior art devices adapted to deliver a tampon into the vaginal orifice is that their use is limited to tampons only, meaning that they are not suitably configured to deliver other forms of insertables, such as, for example, pessaries, suppositories, tablets, sample screening devices, or other devices that are desirable to be deployed into an orifice.

There is a need for improved devices to deploy insertables into an orifice, which preferably offer ease of comfortable, hygienic, and safe, use, that are manufacturable at reasonable cost and in bulk quantities.

Any reference to or discussion of any document, act or item of knowledge in this disclosure is included solely for the purpose of providing a context for the present disclosure. It is not suggested or represented that any of these matters or any combination thereof, formed at the priority date, part of the common general knowledge, or was known to be relevant to attempt to solve any problem with which this disclosure is concerned.

### BRIEF SUMMARY

In this disclosure, a discussion of embodiments of the present disclosure describing the device, should be understood to be embodiments applicable to all aspects of the device of the present disclosure, unless specifically indicated. Even then, persons skilled in the art will readily appreciate which embodiments are adoptable for which aspects of the device of the present disclosure. The same approach is applicable to embodiments of the present disclosure describing method or process aspects, or any other aspects, of the present disclosure.

According to a first aspect, the present disclosure provides a delivery device for deploying an insertable into an orifice guided by digit, the delivery device comprising:
an insertable orientation means having a first longitudinal axis and a base portion, the insertable orientation means adapted to load an insertable positioned in a deployment orientation, and
a skirt member extending from adjacent the insertable orientation means and having a skirt length and comprising opposing digit contacting and orifice contacting surfaces, wherein the skirt member is adapted to be movable substantially parallel to the first longitudinal axis and along the skirt length, between:
   an insertable surrounding configuration, wherein an orifice contacting length of the orifice contacting surface substantially faces the insertable orientation means and any loaded insertable; and
   a digit surrounding configuration, wherein the orifice contacting length of the orifice contacting surface substantially faces away from the insertable orientation means and any loaded insertable.

In some preferred embodiments, a first portion of the skirt member is adapted to form a digit orientation means as the skirt member is moved along the skirt length from the insertable surrounding configuration toward the digit surrounding configuration, the digit orientation means being adapted to orient at least a portion of at least one digit in a navigation orientation adapted to guide deployment of the insertable into the orifice. Preferably, a distal end of the digit orientation means is located on an underside of the insertable orientation means base portion.

In some preferred and some alternative embodiments, the first portion of the skirt member forming the digit orientation means is adjacent a portion of the skirt member interposed between the skirt member first portion and the underside of the insertable orientation means. In some such and other embodiments, the digit orientation means comprises a digit engagement means adapted to temporarily engage at least a portion of a digit adjacent the skirt member first portion. According to such embodiments, the digit engagement means comprises a roughened or otherwise patterned segment of the skirt member first portion. The roughened or patterned segment is preferably adapted to create a substantially fitting engagement between the digit orientation means and the at least a portion of the digit, so as to releasably maintain the digit in the navigation orientation.

The skirt member of preferred embodiments extends from adjacent the insertable orientation means. In some embodiments, the skirt member extends from an outer segment of the insertable orientation means base portion. In others, the skirt member extends from an underside of the insertable orientation means base portion. In some alternative embodiments, the skirt member extends from a distal end of the insertable orientation means.

Preferably, adjacent to where the skirt member extends from adjacent the insertable orientation means, the skirt member turns about itself such that a first portion of the orifice contacting length can, as the skirt member is extended from the insertable surrounding configuration toward the digit surrounding configuration, face, or come into contact with, a second portion of the orifice contacting length. In preferred embodiments, a segment of the skirt member where it turns about itself is an inflection point.

In preferred embodiments, movement of the skirt member along the skirt length from the insertable surrounding configuration toward the digit surrounding configuration commensurately causes a digit contacting length of the digit contacting surface to face, or to come into contact with, at least a portion of at least one digit when the digit is being surrounded by the skirt member. As is discussed below with respect to a method of use aspect of the present disclosure, in some preferred embodiments, an underside of the insertable orientation means base portion is pushed to cause the skirt member to be moved from the insertable surrounding configuration toward the digit surrounding configuration such that the skirt member first portion forms the digit orientation means. In other preferred embodiments, a distal end of the skirt member is pulled so that it moves from the insertable surrounding configuration toward the digit surrounding configuration such that the skirt member first portion forms a digit orientation means. In yet still further preferred embodiments, the skirt member first portion forms a digit orientation means as the underside of the insertable orientation means base portion is pushed and the skirt member is pulled such that the skirt member is moved from the insertable surrounding configuration toward the digit surrounding configuration.

Preferably, movement of the skirt member along the skirt length from the insertable surrounding configuration toward the digit surrounding configuration commensurately causes a first portion of the orifice contacting length of the orifice contacting surface to face, or to come into contact with, a second portion of the orifice contacting length of the orifice contacting surface. In some preferred embodiments, this occurs as the skirt member moves about its inflection point. In preferred and some alternative embodiments, as the skirt member increasingly approaches the digit surrounding configuration, at least a portion of the orifice contacting surface arrives at a position where it faces, or can come into contact with, an inside of the orifice when any loaded insertable is being inserted into the orifice.

In some preferred embodiments, when the skirt member is in the digit surrounding configuration, substantially all of the digit contacting length of the digit contacting surface faces, or comes into contact with, the at least a portion of at least one digit when the digit is surrounded by the skirt member, and substantially all of the orifice contacting length of the orifice contacting surface can face, or can come into contact with, an inside of the orifice.

In yet still further preferred embodiments, when the skirt member is in the digit surrounding configuration, an orifice facing portion of the insertable orientation means can face, and can come into contact with, an inside of the orifice, and any loaded insertable can face, and can come into contact with, an inside of the orifice. In some such embodiments, the orifice facing portion of the insertable orientation means and the loaded insertable are exposed and ready to cause the insertable to be deployed into the orifice.

As referenced above, movement of the skirt member along the skirt length from the digit surrounding configuration toward the insertable surrounding configuration preferably commensurately causes a first portion of the orifice contacting length of the orifice contacting surface to face, or to come into contact with, a second portion of the orifice contacting length of the orifice contacting surface. In some preferred embodiments, this movement occurs about the inflection point. In preferred and some alternative embodiments, as the skirt member increasingly approaches the insertable surrounding configuration, at least a portion of the orifice contacting surface can face, or can come into contact with, an orifice facing portion of the insertable orientation means, and any loaded insertable.

Preferably, movement of the skirt member along the skirt length from the digit surrounding configuration toward the insertable surrounding configuration causes a digit contacting length of the digit contacting surface to face away from, or to come out of contact with, at least a portion of any digit in the digit orientation means.

When the skirt member is in the insertable surrounding configuration, preferably substantially all of the orifice contacting length of the orifice contacting surface faces, and can come into contact with, an orifice facing portion of the insertable orientation means and any insertable loaded for deployment, and substantially all of the digit contacting length of the digit contacting surface faces away from the insertable orientation means and any insertable loaded for deployment.

Other mechanisms for achieving movement of the skirt member along the skirt member length between the insertable surrounding configuration and the digit surrounding configuration are contemplated by this disclosure. However, the following text further describes the mechanisms referenced above.

According to some preferred and some alternative embodiments, following deployment of the insertable into the orifice, the skirt member can be moved from the digit surrounding configuration toward, but short of, the insertable surrounding configuration, adopting a disposable configuration.

Preferably, at least some of any inter-orifice content withdrawn as the delivery device is withdrawn from the orifice may be generally contained between overlapping portions of the orifice contacting surface, located on the orifice contacting surface facing the insertable orientation means, and/or located in and around the insertable orientation means, when the delivery device is in the disposable configuration.

When the delivery device is in the disposable configuration, preferably overlapping portions of the orifice contacting surface and other portions of the orifice contacting surface substantially concentrically surround the digit orientation means and insertable orientation means. In some preferred and some alternative embodiments, a length of the substantially concentric arrangement is between about 1-eighth and about 1-half of a length of the delivery device in the digit surrounding configuration, more preferably between about 1-quarter and about 3-eighths of a length of the delivery device in the digit surrounding configuration. According to some embodiments, the disposable configuration is adapted to facilitate hygienic disposal of the delivery device after insertion of the insertable into the orifice.

When used in this disclosure, the word “hygienic” and grammatical variations of it, used to describe a state of cleanliness means sufficiently clean for medical and paramedical applications, such as, for example, a suitable state of cleanliness for the insertion of a tampon. In some embodiments, the word “hygienic” can also mean an acceptable state of cleanliness for therapeutic goods regulators, such as the Australian Therapeutic Goods Administration and the Australian Pesticides and Veterinary Medicines Authority or the United States Food and Drug Administration, to grant marketing authorization for the sale of insertables adapted to be inserted in an orifice of an animal (including human animal) body without the need for prior sterilization. In that context, “sterilization” and grammatical variations of it, refers to a state of sterility, meaning free of bacteria and other living microorganisms.

In some preferred embodiments, the delivery device further comprises at least one lubrication means along, in association with, or applied to, at least a portion of the skirt length, the lubrication means adapted to facilitate movement of the skirt member along the skirt length between the insertable surrounding configuration and the digit surrounding configuration. Each lubrication means is preferably selected from the group consisting of: an impression formed in a wall of the skirt member, a kink formed in the wall of the skirt member, a raised, depressed, or raised and depressed, pattern formed on the wall of the skirt member, and a lubricating composition applied to a portion of the orifice contacting surface.

Embodiments of the delivery device provide that at least one lubrication means is an impression formed in a wall of the skirt member. In some such embodiments, the impression takes the form of a groove facilitating movement of one portion of the orifice contacting surface over another portion of the orifice contacting surface. Preferably, the impression substantially continuously extends around the skirt member. In one such preferred embodiment, the impression comprises a series of parallel ridges adapted to enable the movement of the skirt member along the skirt length to be achieved by a substantially rotational movement of an end of the skirt member in a spiral direction along the parallel ridges. The impression may take other forms adapted to facilitate movement of skirt member lengths over one another, preferably portions of orifice contacting surface over one another.

Embodiments of the delivery device wherein the at least one lubrication means is comprised of a kink formed in the wall of the skirt member, each kink is preferably located along the skirt member length. In some preferred embodiments, the kink is a substantially S-shape in longitudinal section creating a concave and convex inflection point about which the skirt member can turn. In some such embodiments, by providing further inflection points about which the skirt member can turn, movement of one portion of the orifice contacting surface over another portion of the orifice contacting surface is facilitated. In embodiments wherein the kink is substantially S-shaped, or has other longitudinal section shapes, such as W-shaped or, for example, C-shaped, the introduction of the kink along the skirt length preferably facilitates movement of one portion of the orifice contacting surface over another portion of the orifice contacting surface by having concave and/or convex inflection points along the skirt length. The kink may take other forms adapted to facilitate movement of skirt member lengths over one another, preferably portions of orifice contacting surface over one another.

In some preferred and alternative embodiments, at least one lubrication means comprises a raised, depressed, or raised and depressed pattern formed on the wall of the skirt member. In particularly preferred embodiments, the pattern is formed on an angle essentially preventing inter-engagement of the rises and depressions on a corresponding cross angle pattern when portions of the skirt length are overlapping. In other embodiments, where a lubrication means comprises a raised, depressed, or raised and depressed pattern formed on the wall of the skirt member, and the pattern is irregular. Preferred and alternative embodiments of the raised, depressed, or raised and depressed patterns are selected from the group consisting of: a crosshatched pattern, a spiral pattern, a honeycomb pattern, a pattern comprising a plurality of approximately half sphere-shaped indents in parallel or in a hexagonal layout, and a corrugated pattern. The pattern may take other forms adapted to facilitate movement of skirt member lengths over one another, preferably portions of orifice contacting surface over one another.

In yet still further embodiments, the at least one lubrication means comprises a lubricating composition applied to at least a portion of the orifice contacting surface, the lubricating composition selected from the group consisting of one or more of mineral oils, synthetic oils, solid lubricants, aqueous lubricants, and biolubricants. The lubricating composition is preferably selected according to the application for the delivery device. For example, when the delivery device is being used in a medical or paramedical context, the lubricating composition is comprised of one or more compositions suitable for coming into contact with bodily orifices.

In some preferred and alternative embodiments of the delivery device comprising lubrication means, two or more of the above described, or other, lubrication means are adopted.

The insertable orientation means base portion of preferred embodiments forms a bottom portion of a substantially U-shaped insertable orientation means longitudinal section. Preferably, the opposing arm portions of the substantially U-shape are adapted to provide support for temporary maintenance of the insertable in the deployment orientation. In yet still further preferred embodiments, when the insertable is in the insertable orientation means, a proximal end of the insertable is adjacent, or more preferably comes into contact with, an upper side of the insertable orientation means base portion (at a bottom portion of the U-shape insertable orientation means), such that the proximal end of the insertable is cradled within the U-shape. In some embodiments, some or all of the U-shaped insertable orientation means has a rigidity greater than the rigidity of the skirt member.

As described above, in some preferred embodiments, a first portion of the skirt member forms the digit orientation means. In some such and some alternative embodiments, a longitudinal section of the insertable orientation means and digit orientation means portion of the skirt member has a substantially H-shape. Preferably, the opposing arm portions at the bottom of the H shaped are adapted to provide support for temporary maintenance of the digit in the navigation orientation. In some such embodiments, when a portion of at least one digit is in the digit orientation means, a distal end of the at least one digit is adjacent, or more preferably comes into contact with, an underside of the insertable orientation means base portion, in these embodiments, formed by a top portion of a substantially ∩-shaped component of the substantially H-shape. Preferably, a distal end of the digit is cradled within the ∩-shape. In some embodiments, some or all of the H-shaped longitudinal section of the insertable orientation means and digit orientation means portion of the skirt member has a rigidity greater than the rigidity of the balance of the skirt member.

In some preferred and alternative embodiments adopting the substantially U- and/or substantially H-longitudinal sections described above, instead of the bottom portion of the U-shape or the horizontal portion of the H-shape being substantially straight, the delivery device is formed such that those portions have a substantially S-shape lying down or at an acute angle to the horizontal, or another shape, adapted to offset opposing sides the skirt member relative to one another where the skirt member extends from the insertable orientation means. In some such embodiments, this offsetting has the effect of one opposing side of the skirt member moving before or after the other opposing side of the skirt member when the skirt member is being moved between the insertable surrounding configuration and the digit surrounding configuration.

In some preferred embodiments, the insertable orientation means base portion comprises a partition adapted to separate an insertable oriented for deployment and a digit in the digit orientation means. In embodiments of the delivery device wherein the insertable orientation means is substantially U-shaped longitudinal section, the partition is formed by the bottom of the U-shape. In embodiments of the delivery device wherein the insertable orientation means and digit orientation means have a substantially H-shaped longitudinal section, the partition is formed at the horizontal section of the H-shape. In some preferred and alternative embodiments, the partition is formed as part of the substantially S-shape lying down or at an acute angle to the horizontal, or another shape, adapted to offset opposing sides the skirt member relative to one another where the skirt member extends from the insertable orientation means.

Several preferred and alternative embodiments provide that a cross-section of the insertable orientation means is substantially circular or substantially elliptical. However, the cross-sectional shape of the insertable orientation means is generally determined taking into consideration the cross-sectional shape of the insertable proposed to be loaded into the delivery device for deployment. For example only, when the insertable proposed to be loaded into the delivery device is a tampon or a suppository, the cross-sectional shape of the insertable orientation means will preferably be circular. For further example only, when the insertable proposed to be loaded in the delivery device is a pessary, for example, a doughnut pessary, the cross-sectional shape of the insertable orientation means will preferably be elliptical or otherwise adapted to accommodate the cross-sectional shape of the doughnut pessary.

In preferred embodiments, movement of the skirt between the insertable surrounding configuration and digit surrounding configuration, the skirt member distal end substantially concentrically passes over the insertable orientation means. In alternative embodiments, however, the cross-section of the insertable orientation means can have different shapes, including multilateral, or, for example, square, triangular, polygonal, or star. Embodiments of the orientation means where the cross-section has sharp corners can be further adapted to be rounded at those corners, for example, a square shape can be rounded to have a lipstick case-type cross-section.

Likewise, preferred embodiments of the digit orientation means provide for a multitude of cross-sectional shapes. However, it is particularly preferred for the digit orientation means to have a substantially circular cross-section, which is suited for accommodating at least a portion of at least one digit. This substantially circular cross-section is also preferred for providing substantially controlled and substantially uniform movement of the skirt member between the insertable surrounding configuration and the digit surrounding figuration.

An insertable ejection means adapted to facilitate ejection of the insertable from the insertable orientation means is provided in some preferred and alternative embodiments of the present disclosure. Preferably, the insertable ejection means is positioned so as to be adjacent a distal portion of at least one digit in the digit orientation means. More preferably still, the insertable ejection means is positioned on an underside of the insertable orientation means base portion. In some such embodiments, the insertable ejection means is substantially contained within the digit orientation means. Preferably, the insertable ejection means is a pressable member that can be pressed by a distal portion of at least one digit in the digit orientation means, so as to cause a proximal end of the insertable to be ejected from the insertable orientation means.

In some preferred and alternative embodiments, a portion of the digit orientation means adjacent the insertable ejection means can accommodate a distal length of a fingernail extending beyond the tip of the digit. Similarly, embodiments of the delivery device formed such that the horizontal portion of the U- or H-shaped longitudinal sections described above has a substantially S-shape lying down or at an acute angle to the horizontal, or another shape, adapted to offset opposing sides the skirt member relative to one another where the skirt member extends from the insertable orientation means, can accommodate a distal length of a fingernail extending beyond the tip of the digit in a convex portion of such shape.

The skirt member of some preferred embodiments further comprises an insertable component receiving means adapted to receive a component of the insertable. Such embodiments are particularly preferred for insertables that have a component, which may not be inserted into the orifice. Preferably, the insertable component receiving means is selected from the group consisting of: a skirt member width channel interposed between an opening on the digit contacting surface and an opening on the orifice contacting surface, and a skirt member width slit-type channel opening at a distal end of the skirt member and extending along at least a portion of the skirt length. However, the insertable component receiving means is preferably shaped to accommodate the insertable component. For example, the insertable may include a retrieval string, which itself is not intended to be inserted into the orifice, as is the case with tampons. In such embodiments, the insertable component receiving means is shaped so as to receive at least a portion of a retrieval string connected to, or integral with, the insertable.

Preferred and alternative embodiments of the skirt member have a tapered configuration along the skirt length from substantially widest at the skirt member distal end to substantially narrowest where the skirt member extends from the insertable orientation means. In some such embodiments, the tapering extends from the skirt member distal end along the skirt length to between about 1-eighth and about half of the skirt member length, more preferably between about a quarter and about 3-eighths of the skirt member length, where the tapering ceases. In some embodiments, whether tapered or not, the portion of the skirt member discussed in the previous sentence has a greater rigidity than the balance of the skirt member, preferably between about 0.2 times and about 2 times greater rigidity, more preferably, between about 0.5 times and 1.5 times greater rigidity.

The tapered configuration of some embodiments is adapted to facilitate movement of the skirt member between an insertable surrounding configuration and a digit surrounding configuration, as is a configuration that is not tapered but in which a portion of the skirt member adjacent the skirt member distal end is thicker than a balance of the skirt length. In particularly preferred embodiments, the wider or thicker portion of the skirt member is adapted to be held by a user so as to facilitate the user moving the skirt member between the insertable surrounding configuration and the digit surrounding configuration and, in some embodiments to the disposable configuration. In some such preferred and alternative embodiments, the widest portion of the skirt member thickness is between about 0.1% and about 10.0% thicker than the remainder of the skirt member, more preferably between about 1% and about 8% thicker, and more preferably still between about 2.5% and about 6% thicker.

The insertable orientation means base portion of some preferred embodiments further comprises insertable engagement means adapted to engage at least a proximal end of the insertable. In some such and other embodiments, the insertable orientation means is adapted to compressingly engage with the at least a portion of the insertable, such as the insertable proximal end. In some such embodiments, for example, a shape and/or size of the insertable orientation means is relatively smaller than the at least a portion of the insertable such that the at least a portion of the insertable is temporarily accommodated in the insertable orientation means proximal portion because, during manufacture, or through manual manipulation of a user (for example, on re-loading of the delivery device), the insertable proximal end is forcibly inserted into the insertable orientation means.

Alternatively, the insertable engagement means is sized and shaped so that the insertable proximal end is compressingly maintained in the insertable orientation means. According to some such embodiments, the insertable engagement means comprises at least one rib member. Some such embodiments disclose that engagement can occur through a plurality of rib members. In some embodiments, the plurality of rib members correspond to one or more groove members within the at least a proximal portion of the insertable. Some preferred and alternative embodiments provide that the rib members are equally spaced apart along a perimeter of the insertable orientation means. As would be appreciated by persons skilled in the art, some such embodiments are particularly preferred where the insertable is a tampon as some commercially available tampons are manufactured with groove members equally spaced apart around a perimeter of the tampons.

In embodiments wherein the insertable has an additional component, such as a retrieval string, as is the case with tampons, an orifice may be formed in the insertable orientation means base portion or partition so as to permit passage of a retrieval string (used to enable removal of the tampon from within the vaginal cavity), which typically extends from a proximal portion of commercially available tampons. Embodiments of the delivery device that permit passage of a retrieval string through the insertable orientation means base portion or partition are adapted such that the orifice for receiving the retrieval string has a diameter, or widest portion, only minimally greater than a cross-section of the retrieval string or plurality of retrieval strings. In some preferred embodiments, the material from which the punctured insertable orientation means base portion or partition is formed applies a compressive force onto the retrieval string or plurality of retrieval strings.

As some commercially available tampons have a knot at or adjacent the proximal end of the retrieval string, the diameter, or widest portion of the passageway permitting passage of the retrieval string may be slightly smaller than the knot. In such embodiments, the knot can still be ‘collapsed’ or ‘compressed’ through the passageway upon the provision of a pulling force beyond that which is required to pull the balance of the retrieval string through the passageway. The pulling force sufficient to ‘collapse’ or ‘compress’ the knot through the passageway, and/or to expand the passageway orifice to enable the knot to pass through the base or partition, is facilitated when the tampon has been deployed into the vaginal cavity and maintained there, providing an anchor point for the distal end of the retrieval string against which that pulling force can be applied.

In particularly preferred embodiments, the passageway is configured so that an extent of fluid communication across a thickness of the insertable orientation means base portion or partition is preferably minimized. In some embodiments, the orifice for receiving the retrieval string is sized so as to compressingly maintain the retrieval string from moving without intervention from a user.

In some such preferred embodiments, a distal end of the retrieval string is connected to a proximal end of the tampon, or is interwoven with other materials forming the tampon, such that at least the distal end of the retrieval string remains with the proximal end of the tampon even when the tampon has been deployed into the vaginal cavity. In situ, a proximal end of the retrieval string dangles outside the vagina. A user can grip the distal end of the retrieval string and pull on it to remove the tampon from the vagina when desired.

The digit orientation means of preferred embodiments further comprises a portion adapted to fittingly maintain a portion of at least one digit in the navigation orientation. Preferred and alternative embodiments of the present disclosure disclose that the digit orientation means further comprises a distal portion adjacent the insertable orientation means base portion or partition. In some such embodiments, the digit orientation means distal portion is adapted to fittingly maintain a proportion of a distal phalanx in a digit of a user. In other embodiments, the digit orientation means distal portion can accommodate a length of one or more than one phalanx of a digit.

It is particularly preferred for the digit orientation means distal portion to accommodate at least a portion of one digit. However, some alternative embodiments of the digit orientation means distal portion are further adapted to accommodate at least a portion of more than one digit, for example, two digits or three digits. While it is atypical, in some alternative embodiments, the digit orientation means distal portion is adapted to accommodate four digits or five digits. In some alternative embodiments, the digit is a digit member in the form of a finger. Some such alternative embodiments provide that the digit member is a rod-like apparatus or other similar shaped apparatus manipulable by hand mechanical, or electromechanical means, and can include a robotically controlled digit member.

Preferred and some alternative embodiments provide that the navigation orientation is an orientation in which a portion of at least one digit is oriented for application of at least one directional force to the insertable in the deployment orientation for deployment through physical structures defining the orifice. In some such embodiments, the deployment orientation has a longitudinal axis and the navigation orientation has a longitudinal axis and the arrangement of the deployment orientation and the navigation orientation is such that the deployment orientation longitudinal axis and the navigation orientation longitudinal axis are substantially parallel, substantially aligned, or intersect at an angle adapted to facilitate deployment of the insertable into the orifice.

In some embodiments, the selected alignment of the navigation orientation longitudinal axis and deployment orientation longitudinal axis is further adapted to account for at least one spatially available approach by a hand from which the at least one digit extends to physical structures defining the orifice.

According to some preferred and alternative embodiments, the spatially available approach is an approach that takes account of anthropometric measurements enabling a user to insert the insertable with ergonomic efficiency, meaning from a position that is ergonomically sound and in a timeframe being approximately equivalent to a typical timeframe for insertion of the insertable into an orifice absent the device. In some such embodiments, the expression ‘ergonomically sound’ is one that refers to an approach that does not cause a user to overextend her ordinary range of rotation of the shoulder joint and/or enables the insertable to be deployed into the orifice causing minimal discomfort. A further advantage of this disclosure being particularly suited to ergonomically sound approaches enabling the insertable to be deployed into the orifice causing minimal discomfort is highlighted in circumstances where the insertable is a tampon and a person experiences heightened sensitivity in and around the vagina, particularly associated with penetration, for example, vaginismus.

In some embodiments, the approach encompassed by ‘ergonomically sound’ is that approach that a user self-applying the device to insert a tampon could adopt. For example, a user may adopt an under-hand action to use the device to insert the tampon. In another example, the user may be in a standing position or crouching. In such examples and others, the user will have her thighs spaced apart, and may use her other hand to separate the labia for ease of insertion of the delivery device loaded with the tampon. The approach may include an inward and upward movement of the hand in a curved motion, such that a longitudinal axis of the deployment orientation is angled toward to the lumbar area of the user's spine.

In some embodiments, the insertable is in the deployment orientation when a longitudinal axis of the insertable is substantially parallel to a longitudinal axis of the digit orientation means distal end. In other embodiments, the insertable is in the deployment orientation when the longitudinal axis of the insertable is at an angle to the longitudinal axis of the digit orientation means distal end. In preferred and alternative variations to this embodiment, a physical relationship between the insertable orientation means and the digit orientation means is configured so that a side view of the delivery device reflects an extension of a natural flexed curvature of a user's digit, particularly the natural flexed curvature of the index finger.

In other particularly preferred embodiments, the portion of the least one digit is in the navigation orientation when a longitudinal axis of the at least one digit is substantially parallel to a longitudinal axis of the insertable orientation means proximal end. In some of those embodiments and in others, the at least one directional force is substantially aligned with a central axis of the orifice.

It is particularly preferred for the navigation orientation to orient the at least one digit for application of a plurality of directional forces to the insertable in the deployment orientation, the plurality of directional forces adapted so as to guide the insertable through physical structures defining the orifice and/or to guide the insertable into the orifice.

In preferred embodiments, the delivery device is adapted so that advancement of the insertable through physical structures defining the orifice transmits proprioceptive and tactile cues through the insertable orientation means base portion, or the partition, to a portion of at least one digit enabling informed selection of a directional force or forces necessary to navigate the insertable through physical structures defining the orifice and/or into the orifice. The transmission of the proprioceptive and tactile cues through the insertable orientation means base portion or partition preferably occurs in some embodiments because the navigation orientation is such that the at least a portion of the digit presses up against the partition.

In particularly preferred embodiments, however, proprioceptive and tactile cues are also transmitted to the user's at least one digit through the skirt member. This is because, in such embodiments, at least a portion of the skirt member surrounding the digit has a thickness that enables a user to feel through to the physical structures defining the orifice, acting like a glove interposed between the user's digit and such physical structures.

As would be appreciated by persons skilled in the art, the orifice into which the insertable is deployed is an orifice internal to an animal, including human and non-human animals. Some embodiments of the delivery device are adapted so that the orifice internal to the animal is accessible through an external orifice of the animal. In both preferred and alternative embodiments, the orifice is an opening into a vagina, an opening into an anus, an opening into a urinary tract, an opening into a penis, a nostril, an opening into an ear, a mouth. In some alternative embodiments, the orifice is an opening into a wound or incision.

The delivery device of preferred and alternative embodiments is adapted to deploy various kinds of insertables. In some such embodiments, the insertable is a tampon, pessary, suppository, enema, delivery device for delivering a liquid, cream and/or lubricant, clinical screening device, swab or swabbing device, cream, lubricant, tablet, capsule, caplet, pellet, supplement, implantable medical or surgical device, therapeutic, or other insertable adapted to be of therapeutic or diagnostic benefit. In yet still further embodiments, the insertable may be an active or passive implantable or a device such as a sensor for monitoring, alerting, recording or communicating data recorded from within the orifice, such as biological data, or an implantable therapeutic device for ongoing treatment or medical intervention as required.

In some preferred embodiments, the delivery device can be a single use device, meaning that the delivery device is disposed of following deployment of the insertable into the orifice. Such embodiments suited, for example, to embodiments in which the delivery device and insertable assembly is packaged and/or marketed as a single unit. However, the delivery device can also be a multi-use device. In some such embodiments, whether the delivery device is packaged and/or marketed with or without an insertable or an insertable loaded in the insertable orientation means, a user can wash the delivery device between uses after the insertable has been deployed. When an insertable next needs to be deployed, the user can insert the insertable in the insertable orientation means, and then continue as per the previous occasion.

Materials for the manufacture of preferred embodiments of the delivery device are selected such that they exhibit one or more of the following properties: medical grade, moldable, invertible including invertible without split, non-porous, flexible, resilient, durable, stable at room and transport temperatures, water-soluble, rapidly biodegradable, nonallergenic, approved by therapeutic regulators for medical or ingested drug delivery, and having a shelf-life that exceeds 2 years, more preferably 3 years, and more preferably still, 5 years, when stored according to storage instructions. In some preferred embodiments, another characteristic for the materials is that they do not decompose or melt when the delivery device is in direct sunlight or in a location where the temperature exceeds normal room temperature.

In particularly preferred embodiments, one or more materials of which the delivery device is formed comprise thermoplastic elastomers or moldable bioplastic elastomers including biodegradable bioplastic thermoplastic elastomers.

Some other materials that may be used to manufacture the delivery device include gelatin or potato starch, gel caps, plasticizers, such as glycerin and sorbitol. Coloring or flavoring agents may also be incorporated into the manufacture of the delivery device.

Some specific materials from which the delivery device can be formed include, but are not limited to those selected from the group consisting of: Kuraray Co Ltd's MOWIFLEX™, Mitsubishi Chemical Corporation's BioPBS™, Bio-tec Biologische Naturverpackungen GmbH & Co. KG's BIOPLAST™TPS, Zhejiang Hisun Biomaterials Co Ltd's REVODE213™, Mitsubishi Chemical Corporation's ZELAS™, one or more products from Mitsubishi Chemical Corporation's TEFABLOC™ TOFI210 series, and a styrene block copolymer, including but not limited to, one or more of Kraton Polymers Research B.V.'s THERMOLAST™ M compounds.

In some preferred embodiments, the delivery device is made from a water-dissolvable material. In a particularly preferred embodiment, the device is made from a polyvinyl alcohol polymer (PVA/PVOH). In some preferred and alternative embodiments, the delivery device is made from one or more materials that comprise bioplastic elastomers including biodegradable thermoplastic elastomers.

In some preferred embodiments, the materials from which the delivery device is manufactured are flushable materials. According to INDA/EDANA Guidance Document, flushable materials can be selected from: polyvinyl alcohol resin with a filling agent, a natural polymer material such as carbohydrate, e.g., starch or cellulose, and a plasticizer such as polyols, e.g., glycerol or sorbitol. The polyvinyl alcohol resin raw material may consist of a low alcoholysis degree polyvinyl alcohol resin having an alcoholysis degree of about 80 mol % to 93 mol % and a high alcoholysis degree polyvinyl alcohol resin having an alcoholysis degree of about 93.1 mol % to 99.99 mol %, and the ratio of the low alcoholysis degree polyvinyl alcohol resin to the high alcoholysis degree polyvinyl alcohol resin is from about 90:10 to 0:100 (w/w).

Suitable materials for forming the delivery device are also discussed further below in relation to the manufacturing die and manufacturing process aspects of the present disclosure.

According to a second aspect, the present disclosure provides a method for deploying an insertable into an orifice guided by digit, the method comprising:
a. providing a delivery device according to a first aspect of the present disclosure, wherein an insertable is loaded in the insertable orientation means positioned in a deployment orientation;
b. if the skirt member is:
   i. in the insertable surrounding configuration, advancing a portion of at least one digit against an underside of the insertable orientation means base portion such that a first portion of the skirt member forms a digit orientation means adapted to orient the at least a portion of the at least one digit in a navigation orientation adapted to guide deployment of the insertable into the orifice; or
   ii. between the insertable surrounding configuration and the digit surrounding configuration, or is in the digit surrounding configuration, advancing a portion of at least one digit into the digit orientation means such that the digit is in the navigation orientation;
c. if not already in the digit surrounding configuration, causing the skirt member to adopt the digit surrounding configuration, thereby exposing the insertable;
d. using the at least one digit, guiding deployment of the insertable into the orifice; and
e. withdrawing the delivery device, leaving the insertable deployed in the orifice.

The method of the second aspect of the present disclosure where, following withdrawal of the delivery device, the method further comprises moving the skirt member from the digit surrounding configuration toward, but short of, the insertable surrounding configuration, wherein at least some of any inter-orifice content withdrawn from the orifice is generally contained between overlapping portions of the orifice contacting surface, located on the orifice contacting surface facing the insertable orientation means, and/or located in and around the insertable orientation means.

The method of the second aspect of the present disclosure where, following withdrawal of the delivery device, the digit is withdrawn from the digit orientation means.

The method of the second aspect of the present disclosure wherein the insertable is a tampon with a retrieval string extending from a proximal tampon end and the orifice is a vaginal orifice, and wherein the step of withdrawing the delivery device includes allowing the retrieval string to exit a distal end of the digit orientation means through a passageway formed in the insertable orientation means base portion, thereby being left extending, or dangling, from the vaginal orifice.

Preferred and alternative embodiments of the method disclose that the step of advancing a portion of at least one digit into the digit orientation means includes adjusting the positioning of the portion of the at least one digit until the portion of the at least one digit is in the navigation orientation. Various configurations of the positioning of the portion of the least one digit have been described variously throughout the present disclosure having regard to different embodiments of the delivery device.

For example, in some embodiments, the insertable is a tampon having a retrieval string that extends from a proximal portion of a body of the tampon and a passageway adapted to accommodate a cross-section of the retrieval string is formed in the insertable orientation means base portion or partition. According to some of those embodiments, a proximal end of the retrieval string is in a freely movable configuration within the digit orientation means, or is in a spaghetti-like configuration adjacent a distal end of the digit orientation means. For embodiments of this kind, when a portion of the least one digit is inserted into the digit orientation means, the retrieval string is compressingly maintained between a portion and/or a side of the portion of the at least one digit and an inner wall of the digit orientation means and/or the distal end of the digit orientation means.

According to some preferred embodiments of the method, the step of using the at least one digit, guiding deployment of the insertable into the orifice includes application of at least one directional force such that the insertable is guided through physical structures defining the orifice. In further preferred embodiments of this step, a user selects the directional force or forces necessary to navigate the insertable through the physical structures defining the orifice and/or into the orifice, responsive to proprioceptive and tactile cues transmitted to a portion of the at least one digit through the insertable orientation means base portion or partition, and/or through portions of the skirt member surrounding the digit.

Further steps and/or or parts of steps associated with the method of the second aspect of the present disclosure have also been discussed above when describing positioning and alignment of at the digit orientation means and the insertable orientation means for deployment of the insertable into the orifice.

According to a third aspect, the present disclosure provides a manufacturing die adapted to mold the delivery device of the first aspect of the present disclosure.

Preferably, the manufacturing die is comprised of at least one core plate, and at least one cavity plate. In alternative embodiments, more than one core and cavity plate may be used for the manufacturing die.

In preferred embodiments, the manufacturing die further comprises at least two gates adapted to receive a molten form of a material to fill the die shape, thereby molding the delivery device. In a particularly preferred embodiment, at least three gates are used.

In some preferred and alternative embodiments, the at least three gates are selectively positioned substantially circumferentially and substantially equally spaced apart about the skirt member a predetermined distance relative to where the skirt member extends from the insertable orientation means. Preferably, a determination of the predetermined distance for the selective positioning of the at least three gates is made by reference to where, between the insertable surrounding configuration and the digit surrounding configuration, the die is set to mold the delivery device.

In embodiments where the die is set to mold the delivery device with the skirt member:
in the insertable surrounding configuration, the predetermined distance is preferably between about 0.1% and about 5% of the skirt length;
approximately midway between the insertable surrounding configuration and the digit surrounding configuration, the predetermined distance is preferably between about 5% and about 25% of the skirt length; and
in the digit surrounding configuration, the predetermined distance is preferably between about 95% and about 95.9% of the skirt length.

The predetermined distance for the selective positioning of the at least three gates for embodiments where the die set to mold the delivery device with the skirt member amongst the configurations set out in the 3 bullet points above can be estimated by a person skilled in the art making reference to the information in those 3 bullet points.

In alternative embodiments, different numbers of gates can be used in different locations about the manufacturing die. The number of gates and suitable locations for them are preferably identified by reference to equipment and machinery available in a particular manufacturing plant, and with a view to delivering a mold that meets the description of the delivery device of the first aspect of the present disclosure. In one alternative embodiment, for example only, the at least three gates are selectively positioned adjacent an end of the skirt member.

Preferred and alternative embodiments of the manufacturing die are adapted to mold the delivery device from a material suitable for the application to which the delivery device is to be applied. In particularly preferred embodiments, the material has a Shore Hardness on Shore Hardness Scale A with a value between about 20 and about 60. For embodiments of the delivery device adapted to be used in medical or paramedical applications, the material used will preferably also have biocompatible certification.

The material for forming the delivery device is preferably selected from the group consisting of: thermoplastic elastomers (TPE), thermoplastic urethanes (TPU), polyvinyl alcohol (PVA or PVOH), silicon or silicon equivalents, and other bioplastics or other materials with substantially similar performance characteristics to any of these. However, as discussed above, there are other materials suitable for forming the delivery device of the first aspect of the present disclosure.

According to a fourth aspect, the present disclosure provides a delivery device molded using the manufacturing die of the third aspect of the present disclosure.

According to a fifth aspect, the present disclosure provides a manufacturing jig for assembling a delivery device according to the first aspect of the present disclosure and an insertable, the manufacturing jig comprising an insertable loading means adapted to load the insertable in the insertable orientation means.

Preferably, the insertable loading means comprises:
a. a delivery device supporting member adapted to support the delivery device oriented to receive the insertable loaded into the insertable orientation means; and
b. an insertable supporting member adapted to support the insertable for loading into the insertable orientation means.

Preferred and alternative embodiments of the manufacturing jig provide that components of the jig are in a substantially single track-like arrangement. Preferably, the insertable supporting member is situated distal to the delivery device supporting member.

In some preferred embodiments, insertable has a longitudinal axis and a retrieval string extending substantially from the insertable, and the insertable supporting member is adapted to support the insertable such that an insertable longitudinal axis is substantially aligned with the insertable orientation means first longitudinal axis with the insertable proximal end substantially facing the insertable orientation means. In some such embodiments, the insertable is a tampon.

The manufacturing jig of some preferred and alternative embodiments adapted to facilitate loading of an insertable with a retrieval string, further comprises a retrieval string pulling means with a longitudinal axis and adapted to engage a retrieval string proximal end for pulling through a passageway formed in the insertable orientation means base portion or partition. In some such embodiments, the retrieval string pulling means comprises a string engagement means to which the retrieval string proximal end can be engaged.

Preferably, the retrieval string pulling means longitudinal axis is configured so as to be substantially aligned with the insertable orientation means first longitudinal axis.

In preferred and alternative embodiments, the retrieval string pulling means is further adapted to form the passageway in the insertable orientation means base portion or partition. Some such embodiments provide that the retrieval string pulling means further comprises a puncture means adjacent the string engagement means. The puncture means of preferred and some alternative embodiments is a sharp pointed member capable of penetrating the orientation means base portion or partition.

In some preferred embodiments, the cross-sectional shape of the retrieval string pulling means, including the puncture means, is substantially the same as a cross-section of the retrieval string. Preferably, the cross-section of the retrieval string pulling means is sized for fitting, but releasable, engagement of the retrieval string. In other preferred embodiments, the puncture means produces a single point puncture or a slit puncture that closes up on the inserted retrieval string or strings to apply retaining forces and preferably minimizes possible contact between the digit and the insertable.

In the track-like arrangement, and in other arrangements, the retrieval string pulling means is preferably situated proximal to the delivery device supporting member. In some preferred embodiments, the retrieval string pulling means is adapted to be movable along the retrieval string pulling means longitudinal axis, and wherein as the retrieval string pulling means is advanced toward the delivery device supporting means, the puncture means forms the passageway in the insertable orientation means base portion or partition and extends toward the insertable supporting member.

In preferred embodiments, the retrieval string pulling means pulls the retrieval string proximal end through the passageway in the insertable orientation means base portion or partition upon movement of the retrieval string pulling means away from the insertable orientation means.

In some embodiments, the engagement of the retrieval string proximal end with the string engagement means is achieved by the retrieval string proximal end being tie-ingly engaged to a portion of the retrieval string pulling means. In alternative embodiments, the retrieval string proximal end can be engaged to the string engagement means by hooking engagement, by glue dot engagement, or by any other form of engagement that would enable the retrieval string proximal end to maintain engagement with the string engagement means as the puncture means pulls the retrieval string back through the formed passageway in the insertable orientation means base member or partition.

As the retrieval string pulling means pulls a retrieval string back through the formed passageway in the insertable orientation means base member or partition, the insertable proximal end is preferably engaged with the insertable orientation means, thereby loading the insertable in the insertable orientation means. In preferred embodiments, after the insertable is loaded in the insertable orientation means, the retrieval string proximal end is disengaged from the retrieval string pulling means.

In further preferred and some alternative embodiments, the manufacturing jig of the fifth aspect of the present disclosure further comprises retrieval string placement means adapted to place a length of the retrieval string, that has been pulled through the passageway, adjacent to a distal end of the digit orientation means.

In some preferred embodiments, the length of retrieval string placed adjacent a distal end of the digit orientation means takes a spaghetti-like configuration. Preferably, the retrieval string placement means comprises a source of air adapted to blow the length of retrieval string adjacent the distal end of digit orientation means. However, in alternative embodiments, the retrieval string placement means can take other forms such as, for example, a guiding member adapted to guide the length of retrieval string to adjacent the distal end of the digit orientation means.

According to a sixth aspect, the present disclosure provides a manufacturing process for manufacturing a delivery device and insertable assembly, the manufacturing process comprising:
a. providing a manufacturing die according to the third aspect of the present disclosure, an insertable, a material from which the delivery device is to be formed, and a manufacturing jig according to the fifth aspect of the present disclosure;
b. heating the manufacturing die to a temperature to maintain the material in a molten form, and introducing the material in molten form into the manufacturing die;
c. cooling the manufacturing die such that the molten material solidifies forming the delivery device, and removing the die;
d. loading the formed delivery device and insertable into the manufacturing jig; and
e. using the manufacturing jig to cause the insertable to be loaded in the insertable orientation means.

The manufacturing process of the sixth aspect of the present disclosure, wherein the step of introducing the molten material into the manufacturing die is done under pressure sufficient to cause the molten material to fill the die uniformly.

The manufacturing process of the sixth aspect of the present disclosure further comprising packaging the delivery device and insertable assembly. Preferably, the packaging is formed of biodegradable material, and may also be configured to contain the delivery device and insertable assembly so as to substantially prevent ingress of contaminants.

In some preferred embodiments, the process of manufacture of the sixth aspect of the present disclosure uses process flow techniques with individual process stations arranged on a rotary table, or along a linear conveyor, or robotic arms are employed to perform manufacturing tasks to a defined delivery device molding.

According to a seventh aspect, the present disclosure provides a delivery device and insertable assembly manufactured by the manufacturing process of the sixth aspect of the present disclosure.

### BRIEF DESCRIPTION OF THE DRAWINGS

The present disclosure is explained in detail below with reference to the drawings, in which:

FIG. 1 is a sectional view of a delivery device according to one embodiment of the present disclosure loaded with a tampon. The skirt member of the delivery device is between the insertable surrounding configuration and the digit surrounding configuration;

FIG. 2 is a sectional view of the delivery device shown in FIG. 1, with the skirt member having been moved in the direction of the digit surrounding configuration;

FIG. 3 is a is a sectional view of the delivery device shown in FIG. 1, with the skirt member substantially in the digit surrounding configuration;

FIG. 4 is a is a sectional view of the delivery device shown in FIG. 1, depicting the tampon having been released from the insertable orientation means;

FIG. 5 is a perspective view of a delivery device according to one embodiment of the present disclosure, looking into the skirt member toward the insertable orientation means. There is no insertable depicted in this view;

FIG. 6 is a perspective view of the delivery device shown in FIG. 5, looking into the skirt member toward the digit orientation means;

FIG. 7 is a perspective view of the delivery device according to one embodiment of the present disclosure, looking into the skirt member toward the insertable orientation means. The insertable depicted in this view is a tampon;

FIG. 8 is a perspective view of the delivery device shown in FIG. 7 with a tampon loaded, looking into the skirt member toward the digit orientation means;

FIG. 9 is a sectional view of a delivery device according to one embodiment of the present disclosure loaded with a tampon and bearing a lubrication means according to one embodiment of the present disclosure in the form of an ‘S’-shaped kink formed in the wall of the skirt member;

FIG. 10 is a sectional view of the delivery device shown in FIG. 9. The delivery device depicted includes an insertable ejection means according to one embodiment of the present disclosure;

FIG. 11 is a sectional view of the delivery device shown in FIG. 9, illustrating a behavior of the skirt member;

FIG. 12 is a is a sectional view of the delivery device shown in FIG. 9, with the skirt member having been moved in the direction of the digit surrounding configuration;

FIG. 13 is a is a sectional view of the delivery device shown in FIG. 9, with the skirt member substantially in the digit surrounding configuration;

FIG. 14 is a perspective view of the delivery device according to one embodiment of the present disclosure, looking into the skirt member toward the insertable orientation means. The insertable depicted in this view is a tampon;

FIG. 15 is a perspective view of the delivery device shown in FIG. 14 with a tampon loaded, viewing the skirt member toward the digit orientation means;

FIG. 16 is a side view of a delivery device according to one embodiment of the present disclosure, where the skirt member is substantially in the insertable surrounding configuration;

FIG. 17 is a sectional view of the delivery device shown in FIG. 16, bearing a lubrication means according to one embodiment of the present disclosure in the form of a raised and depressed pattern formed on the wall of the skirt member;

FIG. 18 is a magnified view of the lubrication means shown in FIG. 17;

FIG. 19 is a sectional view of a delivery device according to one embodiment of the present disclosure loaded with a suppository. The skirt member of the delivery device is between the insertable surrounding configuration and the digit surrounding configuration;

FIG. 20 is a sectional view of a delivery device according to one embodiment of the present disclosure loaded with a doughnut-shaped pessary. The skirt member of the delivery device is between the insertable surrounding configuration and the digit surrounding configuration;

FIG. 21 is a perspective view of a delivery device according to one embodiment of the present disclosure, depicting inter-orifice content on and around the insertable orientation means following deployment of an insertable into an orifice;

FIG. 22 is a perspective view of the delivery device shown in FIG. 21 with the skirt member in or approaching the disposable configuration;

FIG. 23 is a perspective view of a manufacturing jig according to one embodiment of the present disclosure. The delivery device is not loaded in this figure;

FIG. 24 is a magnified view of a delivery device supporting member according to one embodiment of the present disclosure for the manufacturing jig shown in FIG. 23;

FIG. 25 is a magnified view of a retrieval string pulling means according to one embodiment of the present disclosure for the manufacturing jig shown in FIG. 23;

FIG. 26 is a different perspective view of the manufacturing jig shown in FIG. 23;

FIG. 27 is a magnified view of an engagement portion of the retrieval string pulling means depicted in FIG. 25;

FIG. 28 is a magnified view of a proximal end of an insertable supporting member according to one embodiment of the present disclosure for the manufacturing jig shown in FIG. 23.

FIG. 29 is a perspective view of the manufacturing jig shown in FIG. 23 with the delivery device loaded on the delivery device supporting member shown in FIG. 24;

FIG. 30 is a different perspective view of the manufacturing jig shown in FIG. 23 with a tampon loaded in the insertable support member. The retrieval string pulling means is shown having formed a passageway for the retrieval string in the insertable orientation means base portion, and the retrieval string shown engaged with the retrieval string pulling means;

FIG. 31 is a magnified view of the tampon retrieval string engaged with the retrieval string pulling means;

FIG. 32 is a perspective view of the manufacturing jig shown in FIG. 30 illustrating the retrieval string pulling means having pulled the tampon retrieval string through the passageway in the insertable orientation means base portion. The tampon is not visible because it is located in the insertable orientation means of the delivery device;

FIG. 33 is a magnified view of the manufacturing jig shown in FIG. 32, with the tampon retrieval string disengaged from the retrieval string pulling means;

FIG. 34 is a magnified view of the manufacturing jig shown in FIG. 30, after the delivery device and insertable assembly has been removed from the manufacturing jig. The delivery device loaded with the tampon is illustrated next to the magnified view of the manufacturing jig. The perspective view of the delivery device looks into the skirt member toward the digit orientation means. The tampon retrieval string is depicted in a spaghetti-like form adjacent a distal end of the digit orientation means;

FIG. 35 is a perspective view of a core plate arrangement for a manufacturing die according to one embodiment of the present disclosure. A sprue left over from material entering the gates and a delivery device formed using the mold are respectively shown between relevant core plates in order to depict the molding process; and

FIG. 36 is a perspective view of a cavity plate arrangement for a manufacturing die corresponding to the core plate arrangement of FIG. 35. The leftover sprue and the formed delivery device are again respectively shown between relevant cavity plates in order to depict the molding process.

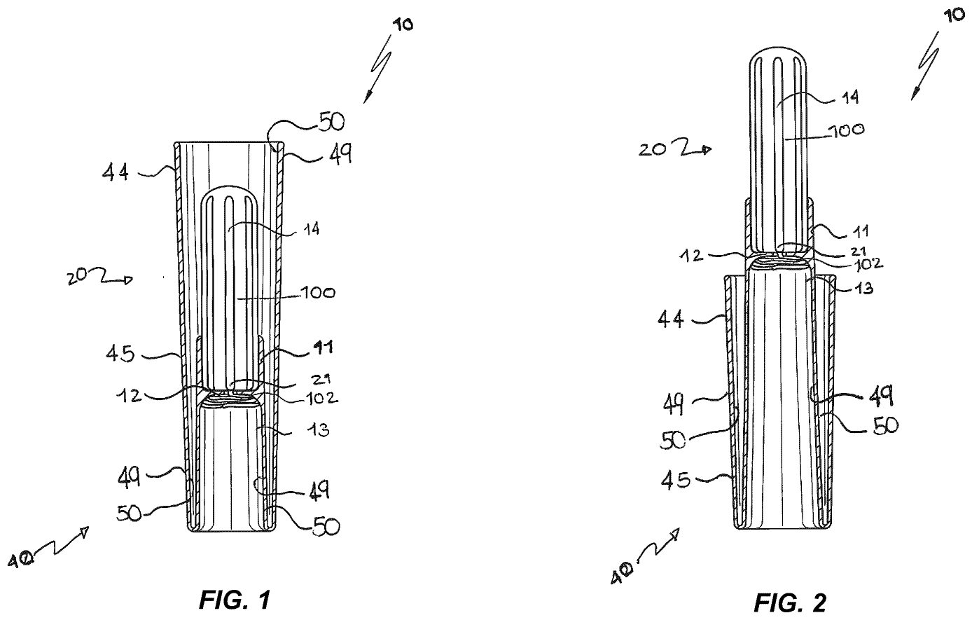

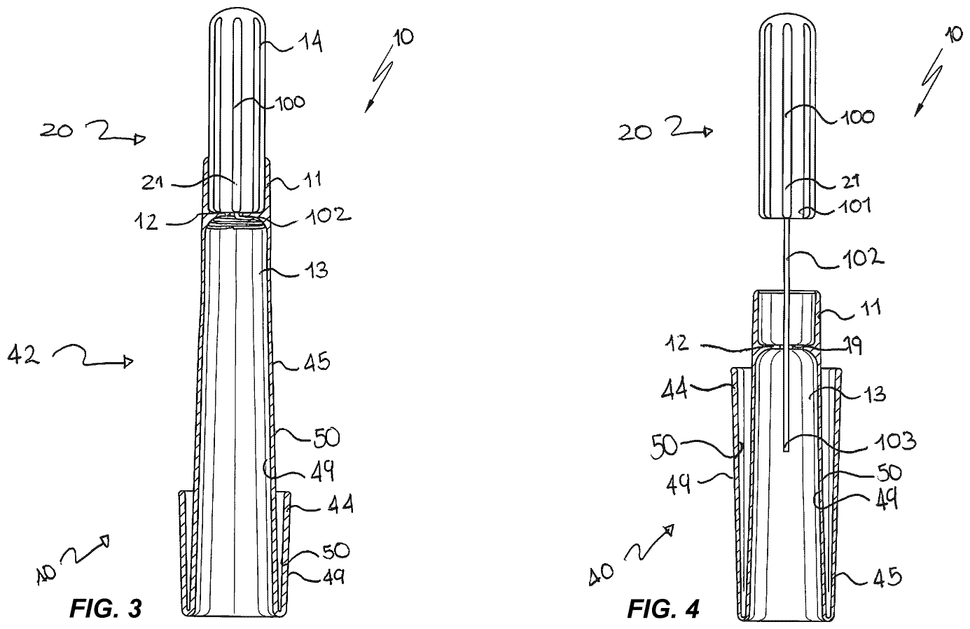

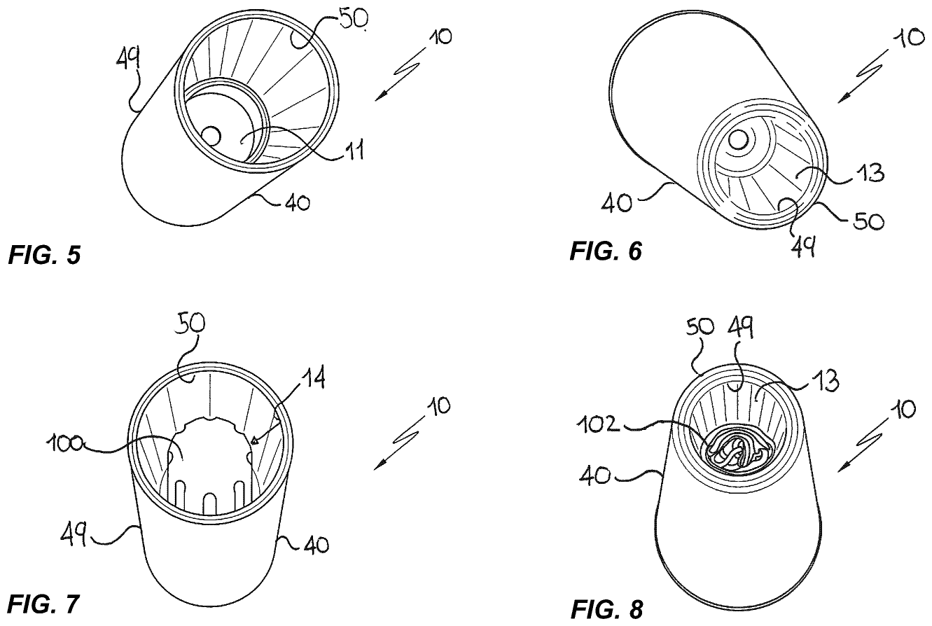

### DETAILED DESCRIPTION

Preferred embodiments of the present disclosure will now be described with reference to the accompanying drawings. While the figures tend to illustrate embodiments in which the insertable or deployable item is a tampon, as has been explained elsewhere in this disclosure, and as would be readily apparent to a skilled addressee, there are many other forms that the insertable or deployable item can take. All such forms are within the scope of the invention.

A delivery device 10 for deploying an insertable 20 into an orifice guided by digit 30, is illustrated in the figures. Broadly, the delivery device 10 comprises:
an insertable orientation means 11 having a first longitudinal axis 15 and a base portion 12, the insertable orientation means 11 adapted to load an insertable 20 positioned in a deployment orientation 14, and
a skirt member 40 extending from adjacent the insertable orientation means 11 and having a skirt length and comprising opposing digit contacting and orifice contacting surfaces 49 and 50, respectively, wherein the skirt member 40 is adapted to be movable substantially parallel to the first longitudinal axis and along the skirt length, between:
   an insertable surrounding configuration 41, wherein an orifice contacting length of the orifice contacting surface 50 substantially faces the insertable orientation means 11 and any loaded insertable 20; and
   a digit surrounding configuration 42, wherein the orifice contacting length of the orifice contacting surface 50 substantially faces away from the insertable orientation means 11 and any loaded insertable 20.

A first portion of the skirt member 40 is adapted to form digit orientation means 13 as the skirt member 40 is moved along the skirt length from the insertable surrounding configuration 41 toward the digit surrounding configuration 42, the digit orientation means 13 being adapted to orient at least a portion of at least one digit 30 in a navigation orientation 16 adapted to guide deployment of the insertable 20 into the orifice. Preferably, a distal end of the digit orientation means 13 is located on an underside of the insertable orientation means base portion 12.

The digit orientation means 13 comprises a digit engagement means adapted to temporarily engage at least a portion of a digit 30 adjacent the skirt member 40 first portion. Preferably, the digit orientation means 13 comprises a roughened or otherwise patterned segment of the skirt member 40 first portion adapted to create a substantially fitting engagement between the digit orientation means 13 and the at least a portion of the digit 30, so as to releasably maintain the digit 30 in the navigation orientation 16.

The skirt member 40 of preferred embodiments extends from adjacent the insertable orientation means 11. Preferably, adjacent to where the skirt member 40 extends from adjacent the insertable orientation means 11, the skirt member 40 turns about itself such that a first portion of the orifice contacting length can, as the skirt member 40 is extended from the insertable surrounding configuration 41 toward the digit surrounding configuration 42, face, or come into contact with, a second portion of the orifice contacting length. A segment of the skirt member 40 where it turns about itself is an inflection point.

Movement of the skirt member 40 along the skirt length from the insertable surrounding configuration 41 toward the digit surrounding configuration 42 commensurately causes a digit contacting length of the digit contacting surface 49 to face, or to come into contact with, at least a portion of at least one digit 30 when the digit 30 is being surrounded by the skirt member 40. An underside of the insertable orientation means base portion 12 is pushed to cause the skirt member 40 to be moved from the insertable surrounding configuration 41 toward the digit surrounding configuration 42 such that the skirt member 40 first portion forms the digit orientation means 13. In other preferred embodiments, a distal end 44 of the skirt member 40 is pulled so that it moves from the insertable surrounding configuration 41 toward the digit surrounding configuration 42 such that the skirt member 40 first portion forms a digit orientation means 13. In yet still further preferred embodiments, the skirt member 40 first portion forms a digit orientation means 13 as the underside of the insertable orientation means base portion 12 is pushed and the skirt member 40 is pulled such that the skirt member 40 is moved from the insertable surrounding configuration 41 toward the digit surrounding configuration 42.

Preferably, movement of the skirt member 40 along the skirt length from the insertable surrounding configuration 41 toward the digit surrounding configuration 42 commensurately causes a first portion of the orifice contacting length of the orifice contacting surface 50 to face, or to come into contact with, a second portion of the orifice contacting length of the orifice contacting surface 50. In some preferred embodiments, this occurs as the skirt member 40 moves about its inflection point. As the skirt member 40 increasingly approaches the digit surrounding configuration 42, at least a portion of the orifice contacting surface 50 arrives at a position where it faces, or can come into contact with, an inside of the orifice when any loaded insertable 20 is being inserted into the orifice.

When the skirt member 40 is in the digit surrounding configuration 42, substantially all of the digit contacting length of the digit contacting surface 49 faces, or comes into contact with, the at least a portion of at least one digit 30 when the digit 30 is surrounded by the skirt member 40, and substantially all of the orifice contacting length of the orifice contacting surface 50 can face, or can come into contact with, an inside of the orifice.

When the skirt member 40 is in the digit surrounding configuration 42, an orifice facing portion of the insertable orientation means 11 can face, and can come into contact with, an inside of the orifice, and any loaded insertable 20 can face, and can come into contact with, an inside of the orifice. In some such embodiments, the orifice facing portion of the insertable orientation means 11 and the loaded insertable 20 are exposed and ready to cause the insertable 20 to be deployed into the orifice. As the skirt member 40 increasingly approaches the insertable surrounding configuration 41, at least a portion of the orifice contacting surface 50 can face, or can come into contact with, an orifice facing portion of the insertable orientation means 11, and any loaded insertable 20.

Movement of the skirt member 40 along the skirt length from the digit surrounding configuration 42 toward the insertable surrounding configuration 41 causes a digit contacting length of the digit contacting surface 49 to face away from, or to come out of contact with, at least a portion of any digit 30 in the digit orientation means 13.

When the skirt member 40 is in the insertable surrounding configuration 41, preferably substantially all of the orifice contacting length of the orifice contacting surface 50 faces, and can come into contact with, an orifice facing portion of the insertable orientation means 11 and any insertable 20 loaded for deployment, and substantially all of the digit contacting length of the digit contacting surface 49 faces away from the insertable orientation means 11 and any insertable 20 loaded for deployment.

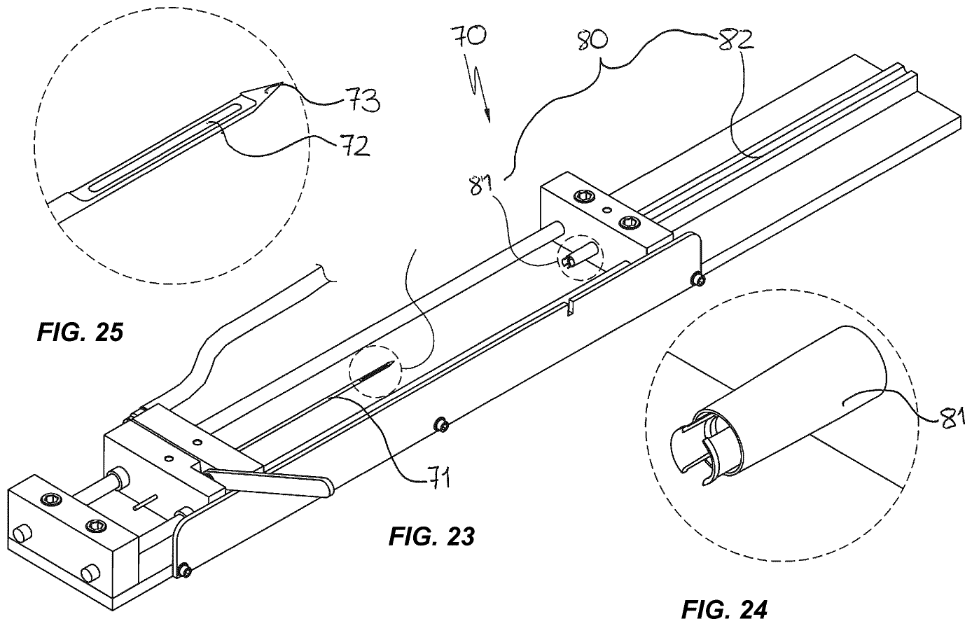

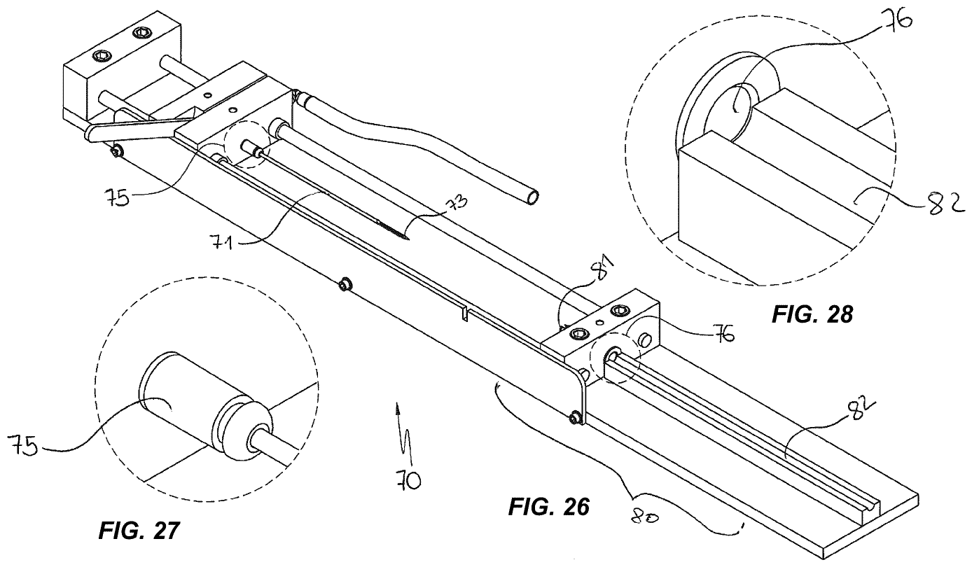

As is illustrated in FIG. 22, following deployment of the insertable 20 into the orifice, the skirt member 40 can be moved from the digit surrounding configuration 42 toward, but short of, the insertable surrounding configuration 41, adopting a disposable configuration 43. Preferably, at least some of any inter-orifice content 48 withdrawn as the delivery device 10 is withdrawn from the orifice may be generally contained between overlapping portions of the orifice contacting surface 50, located on the orifice contacting surface 50 facing the insertable orientation means 11, and/or located in and around the insertable orientation means 11, when the delivery device 10 is in the disposable configuration 43.

When the delivery device 10 is in the disposable configuration 43, preferably overlapping portions of the orifice contacting surface 50 and other portions of the orifice contacting surface 50 substantially concentrically surround the digit orientation means 13 and insertable orientation means 11. A length of the substantially concentric arrangement is between about 1-quarter and about 3-eighths of a length of the delivery device in the digit surrounding configuration 42. The disposable configuration 43 is adapted to facilitate hygienic disposal of the delivery device 10 after insertion of the insertable 20 into the orifice.

The delivery device 10 further comprises at least one lubrication means 18 along, in association with, or applied to, at least a portion of the skirt length, the lubrication means 18 adapted to facilitate movement of the skirt member 40 along the skirt length between the insertable surrounding configuration 41 and the digit surrounding configuration 42. Each lubrication means 18 is preferably selected from the group consisting of: an impression formed in a wall of the skirt member 40, a kink formed in the wall of the skirt member 40, a raised, depressed, or raised and pressed, pattern formed on the wall of the skirt member 40, and a lubricating composition applied to a portion of the orifice contacting surface 50.

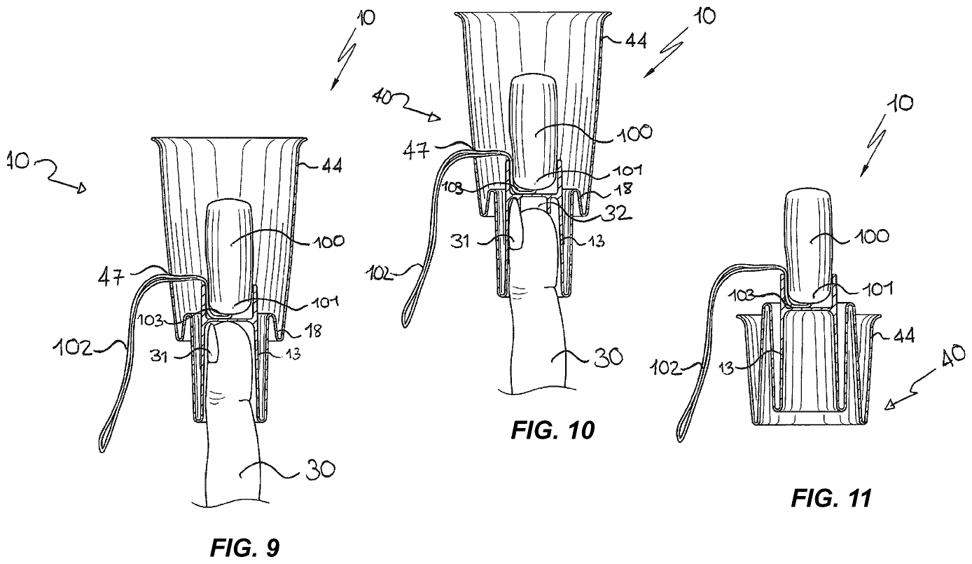

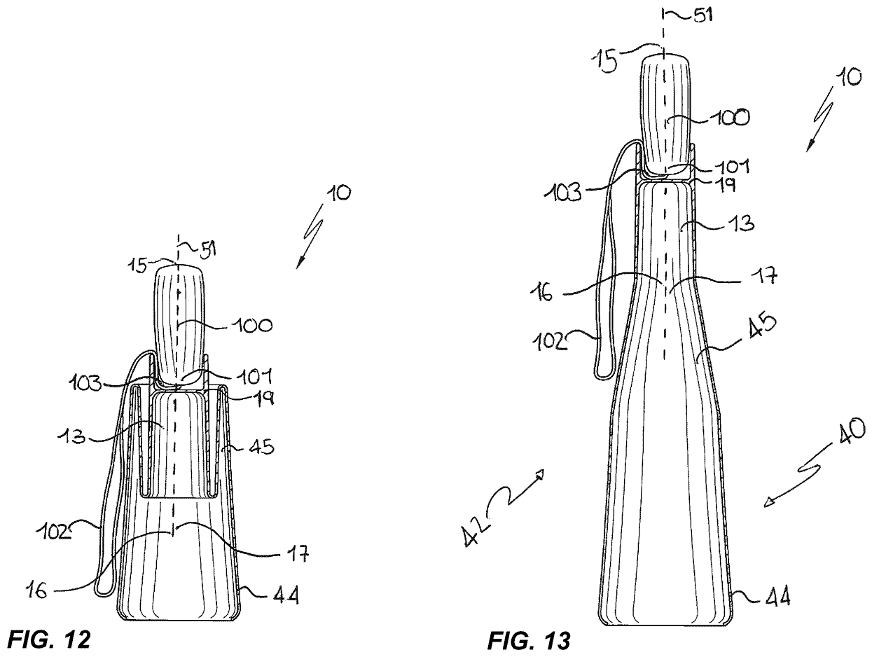

FIGS. 9 to 15 depict embodiments of the delivery device 10 in which one lubrication means 18 is comprised of a kink formed in the wall of the skirt member 40. The kink 18 depicted in the figures is a substantially S-shape in longitudinal section creating a concave and convex inflection point about which the skirt member can turn. By providing further inflection points about which the skirt member 40 can turn, movement of one portion of the orifice contacting surface 50 over another portion of the orifice contacting surface 50 is facilitated.

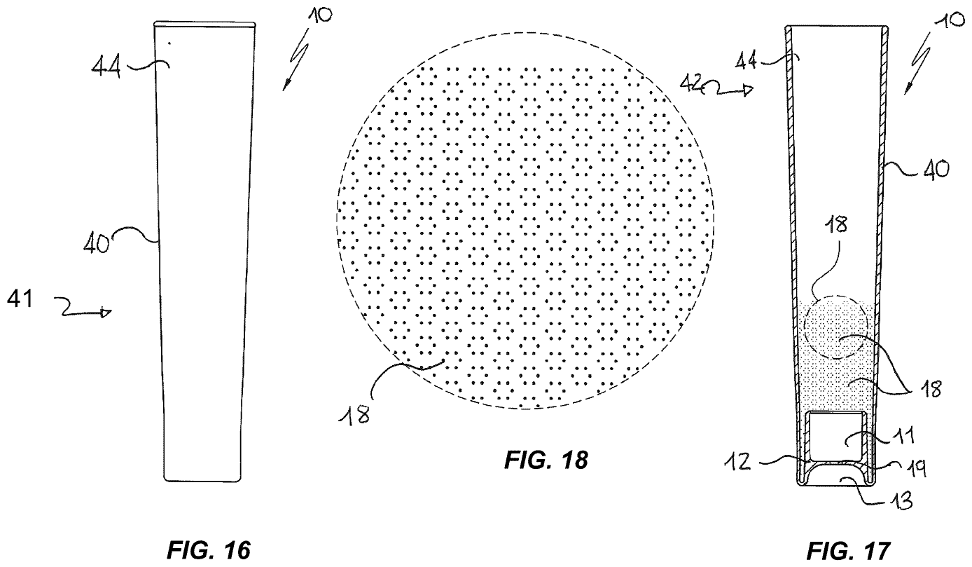

FIGS. 16 to 18 depict embodiments of the delivery device 10 in which one lubrication means 18 is comprised of a raised, depressed, or raised and depressed pattern formed on the wall of the skirt member 40. The pattern 18 of a preferred embodiment is made up of a plurality of half sphere-shaped indents hexagonally arranged and repeated adjacent each wall of each hexagon, is depicted in FIGS. 17 and 18, with 18 being an enlarged view of the pattern.

The insertable orientation means base portion 12 of preferred embodiments forms a bottom portion of a substantially U-shaped insertable orientation means longitudinal section. Preferably, the opposing arm portions of the substantially U-shape are adapted to provide support for temporary maintenance of the insertable 20 in the deployment orientation 14. In yet still further preferred embodiments, when the insertable 20 is in the insertable orientation means 11, a proximal end 21 of the insertable 20 is adjacent, or more preferably comes into contact with, an upper side of the insertable orientation means base portion 14 (at a bottom portion of the U-shaped insertable orientation means 11), such that the proximal end 21 of the insertable 20 is cradled within the U shape. In some embodiments, some or all of the U-shaped insertable orientation means 11 has a rigidity greater than the rigidity of the skirt member 40.

In some preferred embodiments, a longitudinal section of the insertable orientation means 11 and digit orientation means portion 13 of the skirt member 40 has a substantially H-shape. Preferably, the opposing arm portions at the bottom of the H shaped are adapted to provide support for temporary maintenance of the digit 30 in the navigation orientation 16. In some such embodiments, when a portion of at least one digit 30 is in the digit orientation means 13, a distal end of the at least one digit 30 is adjacent, or more preferably comes into contact with, an underside of the insertable orientation means base portion 12, in these embodiments, formed by a top portion of a substantially ∩-shaped component of the substantially H-shape. Preferably, a distal end of the digit 30 is cradled within the ∩-shape. In some embodiments, some or all of the H-shaped longitudinal section of the insertable orientation means 11 and digit orientation means 13 portion of the skirt member 40 has a rigidity greater than the rigidity of the balance of the skirt member 40.

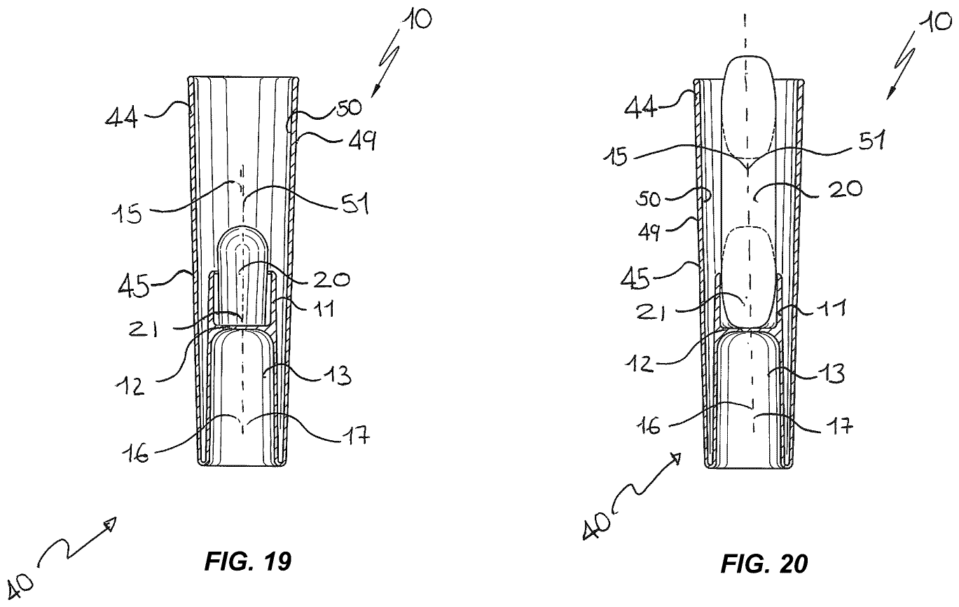

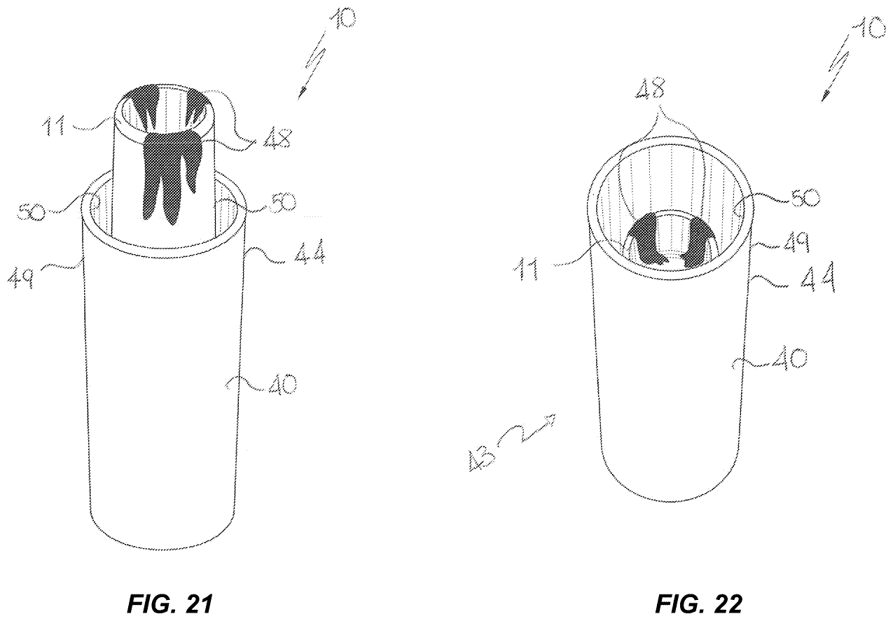

The insertable orientation means base portion 12 of some preferred embodiments comprises a partition 19 adapted to separate an insertable 20 oriented for deployment and a digit 30 in the digit orientation means 13. A cross-section of the insertable orientation means 11 is substantially circular or substantially elliptical. However, the cross-sectional shape of the insertable orientation means 11 is generally determined taking into consideration the cross-sectional shape of the insertable 20 proposed to be loaded into the delivery device for deployment. When the insertable 20 proposed to be loaded into the delivery device 10 is, for example, a tampon 100, or a suppository as depicted in FIG. 19, the cross-sectional shape of the insertable orientation means 11 will preferably be circular. When the insertable 20 proposed to be loaded in the delivery device 10 is a pessary, for example, a doughnut pessary as depicted in FIG. 20, the cross-sectional shape of the insertable orientation means 11 will preferably be elliptical or otherwise adapted to accommodate the cross-sectional shape of the doughnut pessary.

Likewise, preferred embodiments of the digit orientation means 13 provide for a multitude of cross-sectional shapes. However, it is particularly preferred for the digit orientation means 13 to have a substantially circular cross-section, which is suited for accommodating at least a portion of at least one digit 30.

An insertable ejection means 32 adapted to facilitate ejection of the insertable 20 from the insertable orientation means 11 is depicted in FIG. 10. Preferably, the insertable ejection means 32 is positioned so as to be adjacent a distal portion of at least one digit 30 in the digit orientation means 13, on an underside of the insertable orientation means base portion 12. As can be seen in FIG. 10, the insertable ejection means 32 is substantially contained within the digit orientation means 13 and is a pressable member that can be pressed by a distal portion of at least one digit 30 in the digit orientation means 13, so as to cause a proximal end 21 of the insertable 20 to be ejected from the insertable orientation means 11. The insertable ejection means 32 is not depicted in the figures of other embodiments. However, the insertable ejection means 32 can also form part of such other embodiments.

As is shown in FIG. 10, a distal length of a fingernail 31 can be accommodated in a portion of the digit orientation means 13 adjacent an insertable ejection means 32.

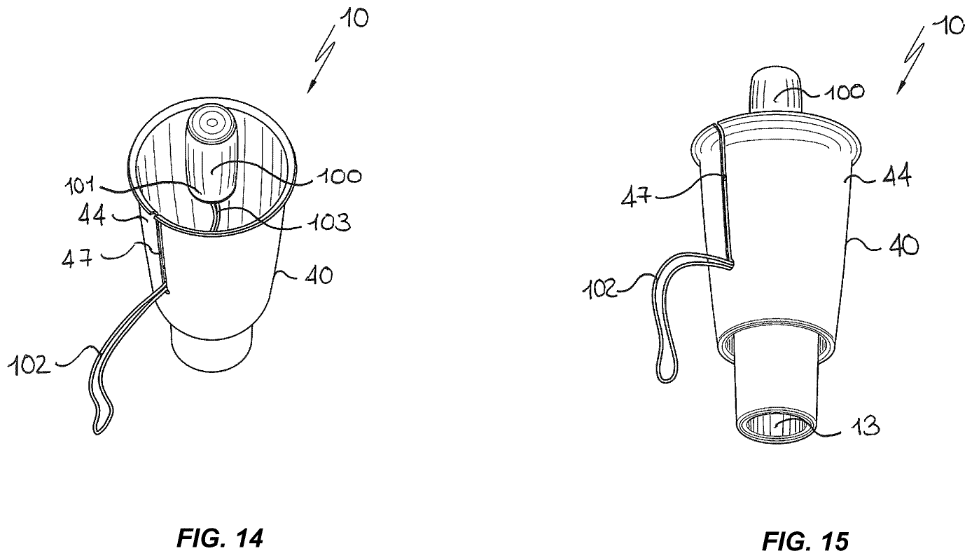

The skirt member 40 of some embodiments further comprises an insertable component receiving means 47 adapted to receive a component of the insertable 20. Some such embodiments are depicted in FIGS. 9 to 15. The insertable component receiving means 47 of one preferred embodiment is depicted clearly in FIGS. 14 and 15 in which it is formed of a skirt member 40 width slit-type channel opening at a distal end 44 of the skirt member 40 and extending along at least a portion of the skirt length.

A shape and/or size of the insertable orientation means 11 is preferably relatively smaller than the at least a portion of the insertable 20 such that the at least a portion of the insertable 20 is temporarily accommodated in the insertable orientation means 11 proximal portion because, during manufacture, or through manual manipulation of a user (for example, on re-loading of the delivery device), the insertable proximal end 21 is forcibly inserted into the insertable orientation means 11.

In embodiments wherein the insertable 20 has an additional component, such as a retrieval string 102, as is the case with tampons 100, an orifice may be formed in the insertable orientation means base portion 12 or partition 19 so as to permit passage of a retrieval string 102 (used to enable removal of the tampon 100 from within the vaginal cavity), which typically extends from a proximal portion 101 of commercially available tampons 100. Embodiments of the delivery device 10 that permit passage of a retrieval string 102 through the insertable orientation means base portion 12 or partition 19 are adapted such that the orifice for receiving the retrieval string 102 has a diameter, or widest portion, only minimally greater than a cross-section of the retrieval string 102. In particularly preferred embodiments, the passageway is configured so that an extent of fluid communication across a thickness of the insertable orientation means base portion 12 or partition 19 is preferably minimized.

The digit orientation means 13 of preferred embodiments further comprises a portion adapted to fittingly maintain a portion of at least one digit 30 in the navigation orientation 16. The navigation orientation 16 is an orientation in which a portion of at least one digit 30 is oriented for application of at least one directional force to the insertable 20 in the deployment orientation 14 for deployment through physical structures defining the orifice. The deployment orientation 14 has a longitudinal axis 15 and the navigation orientation 16 has a longitudinal axis 17 and the arrangement of the deployment orientation 14 and the navigation orientation 16 is such that the deployment orientation longitudinal axis 15 and the navigation orientation longitudinal axis 17 are substantially parallel, substantially aligned, or intersect at an angle adapted to facilitate deployment of the insertable 20 into the orifice.

It is particularly preferred for the navigation orientation 16 to orient the at least one digit 30 for application of a plurality of directional forces to the insertable 20 in the deployment orientation 14, the plurality of directional forces adapted so as to guide the insertable 20 through physical structures defining the orifice and/or to guide the insertable into the orifice, or into a destination position for the insertable 20.

In preferred embodiments, the delivery device 10 is adapted so that advancement of the insertable 20 through physical structures defining the orifice transmits proprioceptive and tactile cues through the insertable orientation means base portion 12, or through the partition 19, to a portion of at least one digit 30 enabling informed selection of a directional force or forces necessary to navigate the insertable 20 through physical structures defining the orifice and/or into the orifice, or into a destination position for the insertable 20. The transmission of the proprioceptive and tactile cues through the insertable orientation means base portion 12 or partition 19 preferably occurs in some embodiments because the navigation orientation 16 is such that the at least a portion of the digit 30 presses up against an underside of the insertable orientation means base portion 12 or against the partition 19.

In particularly preferred embodiments, however, proprioceptive and tactile cues are also transmitted to the user's at least one digit 30 through the skirt member 40. This is because, in such embodiments, at least a portion of the skirt member 40 surrounding the digit 30 has a thickness that enables a user to feel through to the physical structures defining the orifice, acting like a single glove layer interposed between the user's digit and such physical structures.

Also disclosed is a method for deploying an insertable 20 into an orifice guided by digit 30, in which the method comprises:
a. providing a delivery device 10, wherein an insertable 20 is loaded in the insertable orientation means 11 positioned in a deployment orientation 14;
b. if the skirt member 40 is:
   i. in the insertable surrounding configuration 41, advancing a portion of at least one digit 30 against an underside of the insertable orientation means base portion 12 such that a first portion of the skirt member 40 forms a digit orientation means 13 adapted to orient the at least a portion of the at least one digit 30 in a navigation orientation 16 adapted to guide deployment of the insertable 20 into the orifice; or
   ii. between the insertable surrounding configuration 41 and the digit surrounding configuration 42, or is in the digit surrounding configuration 42, advancing a portion of the at least one digit 30 into the digit orientation means 13 such that the digit 30 is in the navigation orientation 16;
c. if not already in the digit surrounding configuration 42, causing the skirt member 40 to adopt the digit surrounding configuration 42, thereby exposing the insertable 20;
d. using the at least one digit 30, guiding deployment of the insertable 20 into the orifice, and
e. withdrawing the delivery device 10, leaving the insertable 20 deployed in the orifice.

Following withdrawal of the delivery device 10, the method further comprises moving the skirt member 40 from the digit surrounding configuration 42 toward, but short of, the insertable surrounding configuration 41, wherein at least some of any inter-orifice content 48 withdrawn from the orifice is generally contained between overlapping portions of the orifice contacting surface 50, located on the orifice contacting surface 50 facing the insertable orientation means 11, and/or located in and around the insertable orientation means 11. Also following withdrawal of the delivery device 10, the digit 30 is withdrawn from the digit orientation means 13.

The step of advancing a portion of at least one digit 30 into the digit orientation means 13 includes adjusting the positioning of the portion of the at least one digit 30 until the portion of the at least one digit 30 is in the navigation orientation 16.

In some preferred embodiments, the insertable 20 is a tampon 100 having a retrieval string 102 that extends from a proximal portion 101 of a body of the tampon 100 and a passageway adapted to accommodate a cross-section of the retrieval string 102 is formed in the insertable orientation means base portion 12 or partition 19. A proximal end 103 of the retrieval string 102 is in a freely movable configuration within the digit orientation means 13, or is in a spaghetti-like configuration adjacent a distal end of the digit orientation means 13. For embodiments of this kind, when a portion of the least one digit 30 is inserted into the digit orientation means 13, the retrieval string 102 is compressingly maintained between a portion and/or a side of the portion of the at least one digit 30 and an inner wall of the digit orientation means 13 and/or the distal end of the digit orientation means 13.

The step of using the at least one digit 30, guiding deployment of the insertable 20 into the orifice includes application of at least one directional force such that the insertable is guided through physical structures defining the orifice. In further preferred embodiments of this step, a user selects the directional force or forces necessary to navigate the insertable 20 through the physical structures defining the orifice and/or into the orifice, or into a destination position within the orifice, responsive to proprioceptive and tactile cues transmitted to a portion of the at least one digit 30 through the insertable orientation means base portion 12 or partition 19, and/or through portions of the skirt member 40 surrounding the digit 30.

Further disclosed is a manufacturing die 60 adapted to mold the delivery device 10. Preferably, the manufacturing die 60 is comprised of at least one core plate 61, and at least one cavity plate 62. The manufacturing die 60 further comprises at least three gates (shown as sprue 63 left behind following molding) adapted to receive a molten form of a material to fill the die 60 shape, thereby molding the delivery device 10.

The at least three gates (shown as sprue 63 left behind following molding) are selectively positioned substantially circumferentially and substantially equally spaced apart about the skirt member 40 a predetermined distance relative to where the skirt member 40 extends from the insertable orientation means 11. Preferably, a determination of the predetermined distance for the selective positioning of the at least three gates is made by reference to where, between the insertable surrounding configuration 41 and the digit surrounding configuration 42, the die 60 is set to mold the delivery device 10.

In embodiments where the die 60 is set to mold the delivery device 10 with the skirt member 40:
in the insertable surrounding configuration 41, the predetermined distance is preferably between about 0.1% and about 5% of the skirt length;
approximately midway between the insertable surrounding configuration 41 and the digit surrounding configuration 42, the predetermined distance is preferably between about 5% and about 25% of the skirt length; and
in the digit surrounding configuration 42, the predetermined distance is preferably between about 95% and about 95.9% of the skirt length.

The manufacturing die 60 of preferred embodiments is adapted to mold the delivery device 10 from a material suitable for the application to which the delivery device is to be applied. In particularly preferred embodiments, the material has a Shore Hardness on Shore Hardness Scale A with a value between about 20 and about 60. For embodiments of the delivery device adapted to be used in medical or paramedical applications, the material used will preferably also have biocompatible certification.

The material for forming the delivery device is preferably selected from the group consisting of: thermoplastic elastomers (TPE), thermoplastic urethanes (TPU), polyvinyl alcohol (PVA or PVOH), silicon or silicon equivalents, and other bioplastics or other materials with substantially similar performance characteristics to any of these. However, as discussed above, there are other materials suitable for forming the delivery device of the first aspect of the present disclosure.

Yet still further disclosed is a delivery device 10 molded using manufacturing die 60.

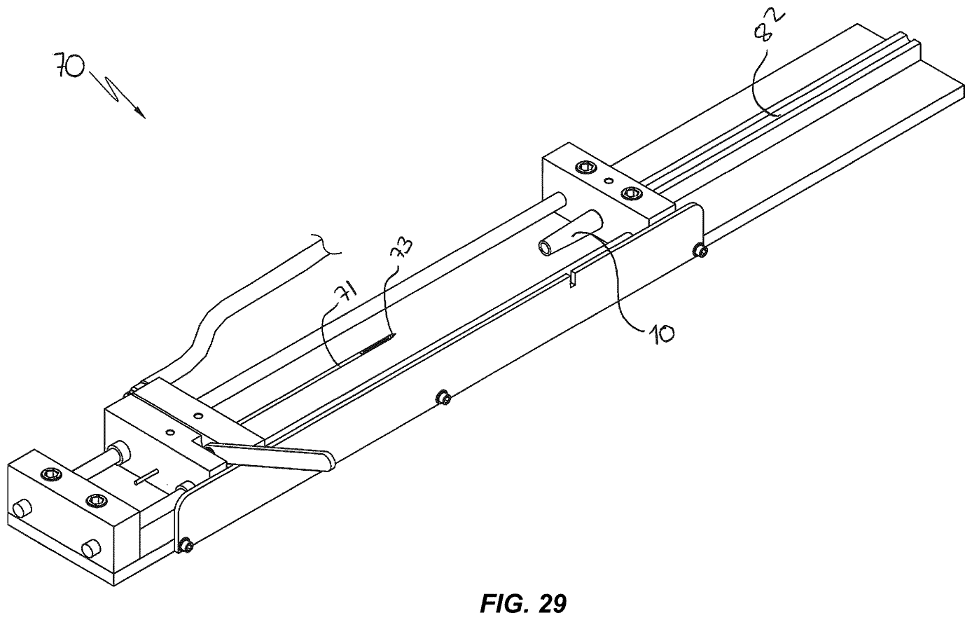

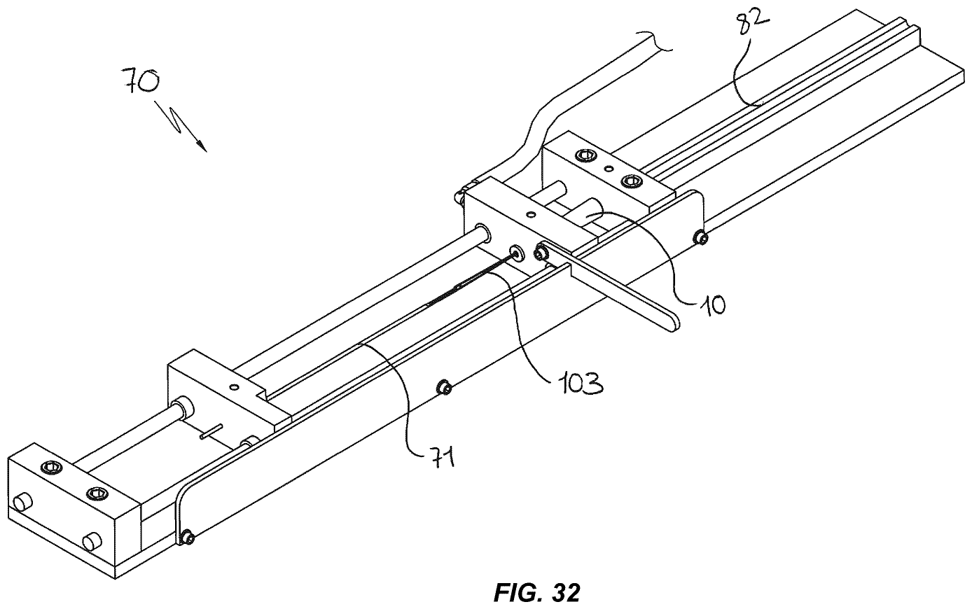

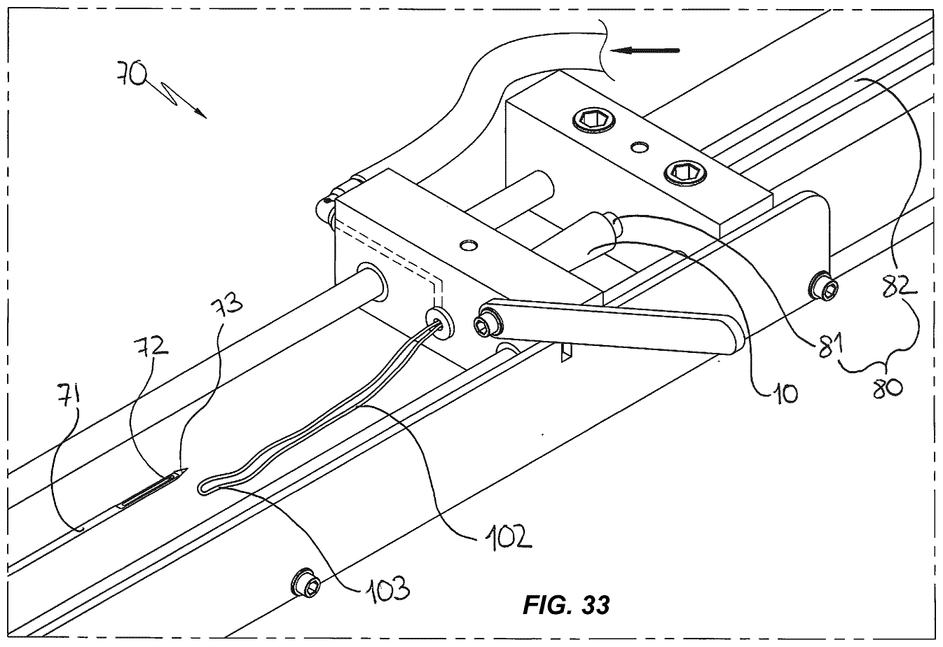

Also disclosed is a manufacturing jig 70 for assembling a delivery device 10 and an insertable 20, depicted in FIGS. 23 to 34. The manufacturing jig 70 comprises an insertable loading means 80 adapted to load the insertable 20 in the insertable orientation means 11. Preferably, the insertable loading means 80 comprises:
a. a delivery device supporting member 81 adapted to support the delivery device 10 oriented to receive the insertable 20 loaded into the insertable orientation means 11; and
b. an insertable supporting member 82 adapted to support the insertable 20 for loading into the insertable orientation means 11.

In a preferred embodiment, components of the jig 70 are in a substantially single track-like arrangement. Preferably, the insertable supporting member 82 is situated distal to the delivery device supporting member 81.

In some preferred embodiments, insertable 20 has a longitudinal axis and a retrieval string 102 extending substantially from the insertable 20, and the insertable supporting member 82 is adapted to support the insertable 20 such that an insertable longitudinal axis is substantially aligned with the insertable orientation means first longitudinal axis 51 with the insertable proximal end 21 substantially facing the insertable orientation means 11. In some such embodiments, the insertable is a tampon 100.

The manufacturing jig 70 adapted to facilitate loading of an insertable 20 with a retrieval string 102, further comprises a retrieval string pulling means 71 with a longitudinal axis and adapted to engage a retrieval string proximal end 103 for pulling through a passageway formed in the insertable orientation means base portion 12 or partition 19. The retrieval string pulling means 71 is fitted to the manufacturing jig 70 by retrieval string pulling means support member 75.

The retrieval string pulling means 71 comprises a string engagement means 72 to which the retrieval string proximal end 103 can be engaged. Preferably, the retrieval string pulling means 71 longitudinal axis is configured so as to be substantially aligned with the insertable orientation means first longitudinal axis 51.

The retrieval string pulling means 71 is further adapted to form the passageway in the insertable orientation means base portion 12 or partition 19. Some such embodiments provide that the retrieval string pulling means 71 further comprises a puncture means 73 adjacent the string engagement means 72. The puncture means 73 is preferably a sharp pointed member capable of penetrating the orientation means base portion 12 or partition 19.

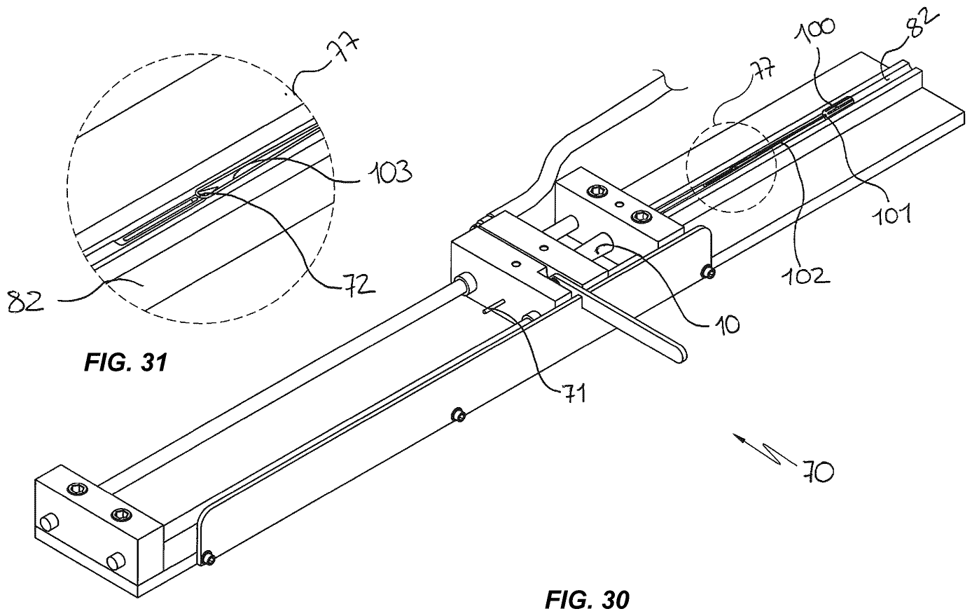

In the track-like arrangement, and in other arrangements, the retrieval string pulling means 71 is preferably situated proximal to the delivery device supporting member 81. In some preferred embodiments, the retrieval string pulling means 71 is adapted to be movable along the retrieval string pulling means longitudinal axis, and wherein as the retrieval string pulling means 71 is advanced toward the delivery device supporting member 81, as depicted in FIG. 30, the puncture means 73 forms the passageway in the insertable orientation means base portion 12 or partition 19 and extends toward the insertable supporting member 82.

In preferred embodiments, the retrieval string pulling means pulls the retrieval string proximal end 103 through the passageway in the insertable orientation means base portion 12 or partition 19 upon movement of the retrieval string pulling means 71 away from the insertable orientation means 11. This is depicted in FIG. 32.

As the retrieval string pulling means 71 pulls a retrieval string 102 back through the formed passageway in the insertable orientation means base member 12 or partition 19, the insertable 20 passes through the insertable receiving orifice 76, depicted in FIG. 28, and the insertable proximal end is subsequently preferably engaged with the insertable orientation means 11, thereby loading the insertable 20 in the insertable orientation means 11. In preferred embodiments, and as is depicted in FIG. 33, after the insertable 20 is loaded in the insertable orientation means 11, the retrieval string proximal end 103 is disengaged from the retrieval string pulling means 71.

Manufacturing jig 70 further comprises retrieval string placement means 74 adapted to place a length of the retrieval string 102, that has been pulled through the passageway, adjacent to a distal end of the digit orientation means 13.

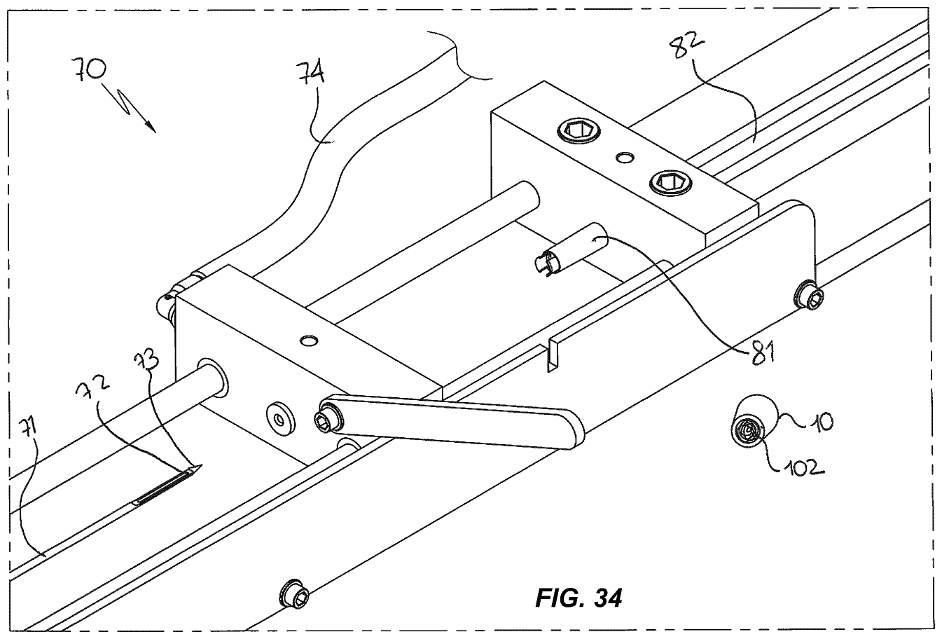

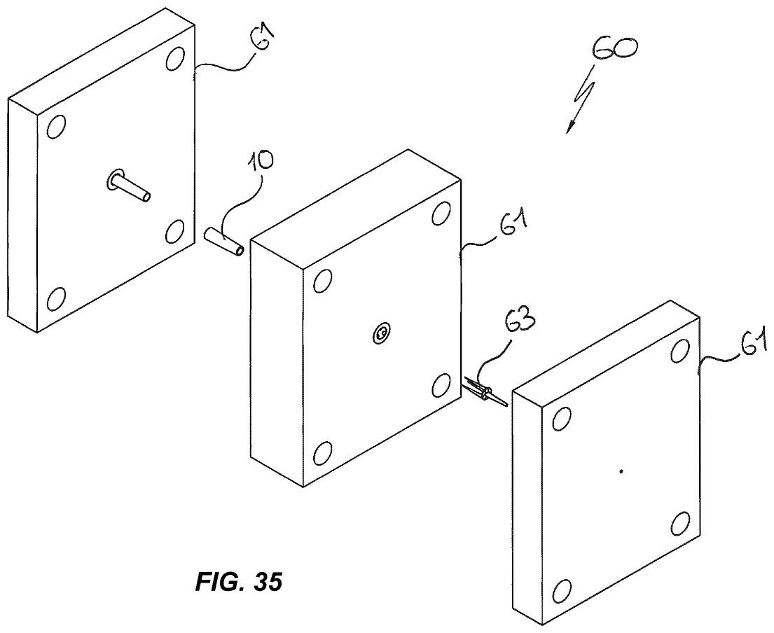

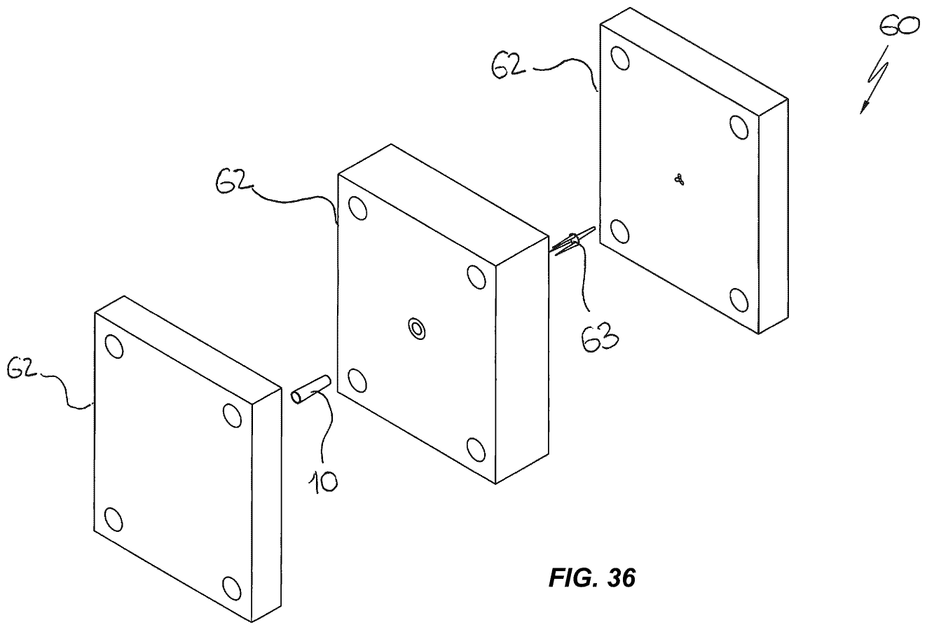

In some preferred embodiments, the length of retrieval string placed adjacent a distal end of the digit orientation means 13 takes a spaghetti-like configuration. This is depicted as 102 in FIG. 34. Preferably, the retrieval string placement means 74 comprises a source of air (not shown) adapted to blow the length of retrieval string adjacent the distal end of digit orientation means 13.

Further disclosed is a manufacturing process for manufacturing a delivery device 10 and insertable 20 assembly, the manufacturing process comprising:
a. providing a manufacturing die 60, an insertable 20, a material from which the delivery device 10 is to be formed, and a manufacturing jig 70;
b. heating the manufacturing die 60 to a temperature to maintain the material in a molten form, and introducing the material in molten form into the manufacturing die 60;
c. cooling the manufacturing die 60 such that the molten material solidifies forming the delivery device 10, and removing the die 60;
d. loading the formed delivery device 10 and insertable 20 into the manufacturing jig 70; and
e. using the manufacturing jig 70 to cause the insertable 20 to be loaded in the insertable orientation means 11.

Preferably, the step of introducing the molten material into the manufacturing die 60 is done under pressure sufficient to cause the molten material to fill the die 60 uniformly.

The manufacturing process further comprises packaging the delivery device 10 and insertable 20 assembly. Preferably, the packaging is formed of biodegradable material.

Moreover, the delivery device 10 could be manufactured a number of ways. Preferably, it is manufactured as a low-cost item and made of a single material without joints in a process that produces multiple delivery devices 10 simultaneously. Single piece manufacture requires the manufacturing die 60 adapted to very shape and thickness in different areas of the ultimately molded delivery device 10. For example only, the flexible structural strength of the distal end 44 of the skirt member 40, insertable orientation means 11, and digit orientation means 13 may require a different thickness to the remainder 45 of the skirt member 40, requiring elasticity and thinness to facilitate movement of the skirt member 40 about the inflection point, with other properties being consistent in thickness in the delivery device 10.

Simple injection molding is a particularly preferred manufacturing methodology for the delivery device 10, as described above. However, some variations in injection molding that could be used include double-shot injection molding and injection molding with over molding (injection). While more complex, and therefore costly, the delivery device 10 could also be manufactured using injection dip molding, or injection blow molding, injection blow molding together with extrusion blow molding, extrusion thermoforming, or forming and welding of calendared elastomeric films. These are described in more detail below with respect to alternative embodiments of the manufacturing process.

In some alternative embodiments of the manufacturing process, for example, the injection molding machine has at least two injectors each with a different material or materials of different grade. In some such embodiments, the H-shaped longitudinal section is manufactured using one softness grade of the selected thermoplastic, while the remainder of the delivery device 10 is made by a shot of a different grade (e.g., softness, color, additive) material formed around the H-shaped longitudinal section, bonding to it substantially permanently. This manufacturing methodology may require custom molding machinery and molds.

In some other alternative embodiments of the manufacturing process, for example, the H-shaped longitudinal section is manufactured using one softness grade of the selected thermoplastic by injection molding. That section of delivery device 10 is then placed into another mold of the skirt member 40 for injection overmolding, where a shot of a different grade (e.g., softness, color, additive) material is injected around the first molded section, bonding to it substantially permanently.

In yet still further alternative embodiments of the manufacturing process, for example, injection blow molding, the H-shaped longitudinal section shape is injection molded with a bulb shape at a delivery end, creating a ‘preform.’ The ‘preform’ is then placed in a custom blow molding machine where the bulb is softened with heat and internal air pressure expands the bulb to fill a shaped mold, which is then cooled before the delivery end is trimmed off, thereby creating an opening to insert the delivery device. Subsequently, this is inverted and ready to fill.

In some further alternative embodiments of the manufacturing process, for example, a shape of a portion of the delivery device 10, such as the skirt member 40, is made in injection molding machine by dip-molding. The H-shaped longitudinal section shape is manufactured in injection molder as a subcomponent that is then fitted to a dip mold form. The dip mold form is dipped into elastomeric thermoplastic so that a portion of the injection molding is dipped to form a bond when cooled. Preferably, the end of the dip molding is trimmed off to create the opening to insert the delivery device 10. Subsequently, this is inverted and ready to fill.

Further alternative embodiments of the manufacturing process provide, for example, that a shape of a portion of the delivery device 10, such as the H-shaped longitudinal section, is formed in an injection molding machine. A shape of another portion of the delivery device 10, such as the skirt member 40, is made by dip-molding. The H-shaped longitudinal section shape and the skirt member 40 shape are assembled. Subsequently this is inverted and ready to fill.

In further alternative embodiments of the manufacturing process, for example, injection molding and extrusion blow molding are adopted. A shape of a portion of the delivery device 10, for example, the H-shaped longitudinal section, is formed in an injection molding machine. Another portion of the delivery device 10, such as the skirt member 40, is made by extruding an elastomeric tube (e.g., medial tubing). The extrusion is preferably cut length and heated, and then compressed air blows the polymer into a shaped mold to create the skirt member 40 shape. The two shape portions are then assembled, and inverted ready to fill.

Further alternative embodiments of the manufacturing process adopt injection molding and extrusion thermoforming. For example, a shape of a portion of the delivery device 10, such as of the H-shaped longitudinal section, is formed using injection molding. A shape of another portion of the delivery device 10, such as the skirt member 40, is made by extruding an elastomeric tube (e.g., medial tubing). The extrusion is preferably cut length, heated and shaped by insertion of a shaped mandrel to form the skirt member 40 shape. The two shape portions are then assembled, and inverted ready to fill.

In yet still further alternative embodiments of the manufacturing process, for example, a shape of the portion of the delivery device 10, such as the H-shaped longitudinal section, is formed in an injection molding machine. A shape of a further section of the delivery device 10, such as the skirt member 40, is made by calendaring an elastomeric sheet (e.g., a thin mat). The sheet is preferably die-cut to shape, heated and formed on a shaped mandrel to form the skirt member 40 shape and heat welded down a seam. The two shape portions are then assembled, and inverted ready to fill.

Yet still further alternative embodiments of the manufacturing process provide for the delivery device 10, preferably in the insertable surrounding configuration 41 and, in some embodiments, with the H-shaped longitudinal section also substantially everted, to be manufactured by digital manufacturing to extrude-print a 3D object layer in suitable materials.

Further disclosed is a delivery device 10 and insertable 20 assembly manufactured by a manufacturing process as described above.

### EXAMPLES

The following exemplary description of the evolution of the delivery device of the present disclosure is now provided, which is not intended to be limiting.

### Example 1

Early designs of a device adapted to be used for deploying an insertable into an orifice identified multiple improvements that would produce a desirable, improved delivery device.

Development work started with a focus on producing a device with multiple features, each with differing, and sometimes conflicting physical requirements. Early designs and production trials were able to achieve these differing requirements in a single product by use of different materials and manufacturing processes for each element having its own feature set. The elements were then combined into a single (multipiece) assembly. However, it was found that this manufacturing approach was intricate and likely to expensive to produce a viable device. It was also found that adopting this manufacturing approach made it difficult to maintain stringent quality control.

### Example 2

A subsequent approach resulted in a redesign of the device as a single mold, preferably maintaining desirable functionality and enabling simpler assembly. The process of injection molding was selected for the multiple designs and production trials according to this Example 2 with a view to mold approved medical grade elastomeric polymers in a one-piece design. Injection molding was thought to be ideal for producing large numbers of identical finished devices, but initially led to limitations associated with maintaining sufficiently thin-walled sections to be used in a sheathing member adapted to enable the insertable 20 to be exposed ready for insertion into the orifice. Identification of appropriate materials became a significant part of the challenge to produce a delivery device 10.

Multiple designs and production trials in this Example 2 established that achieving thin-walled sections produced incomplete moldings, while thicker walled sections impaired both effective inversion and the ability for the user to achieve adequate proprioceptive and tactile cues so as to guide by digit 30 deployment of the insertable 20 into the orifice, and determining correct placement of the insertable within the orifice.

### Example 3

Another subsequent approach adopted was to mold a core socket feature, such as the H-shaped longitudinal section with injection molding, and to extrude and then thermally deform a sheath member (adapted to be movable between an insertable surrounding configuration and a digit surrounding configuration), and bond both elements together in an automated process. Again, multiple designs and production trials were made and conducted according to this approach adopted in Example 3. However, the elastomeric material used for the sheath member could not withstand all of the processes steps being carried out and failed. Identification of appropriate materials continued to remain a significant challenge.

### Example 4

The designs and production trials in Examples 1 to 3 also included a molded top seal that enabled the insertable 20 to be housed hygienically between insertable orientation means 11 and molded top seal, and surrounded by a portion of the sheath member. The molded top seal added complications to material selection and manufacturing process.

Accordingly, a subsequent approach adopted was to abandon inclusion of the molded top seal, and rely instead on packaging to provide an effective seal of the insertable 20 maintained in the insertable orientation means 11. Animated digital simulation was employed to model new open-top designs, taking into consideration qualities of different materials.

Designs and production trials in this Example 4 also sought to adopt variations on the approach to inverting the sheath member, with a view to improving performance of the sheath member when moving between the deployment surrounding configuration and digit surrounding configuration. In some of these designs, the digit orientation means was adapted to push through the middle of the sheath member rather than to have the sheath member pulled down to reveal the insertable 20. However, structural changes to the delivery device to adopt this approach resulted in unsatisfactory performance of inversion. In other of these designs, a new location for an inversion-starter role with a new shape and thickness profile of the sheath member were adopted.

Identifying suitable relative positions of moving parts of the delivery device posed challenges, as did friction between portions of the sheath member moving overlappingly during inversion or reversion.

### Example 5

Several of the materials trialed were found to exhibit strong ‘cling’ characteristics when folded over and overlapping portions slid against one another. Lubricant formulations, such as medical grade lubricants, were applied to surfaces of the sheath member made in designs and production trials for this Example 5, which would, during inversion or reversion, slide over one another. While this did improve the ability for such surfaces to slide over one another, it created an undesirable level of complexity both because keeping the lubricant formulation on the relevant surface of the device when in packaging could prove difficult, if not possible, and adding a lubricant formulation to the delivery device 10 created further manufacturing steps and, possibly, additional manual steps for the user.

It will be appreciated by persons skilled in the art that numerous variations and/or modifications may be made to the invention as shown in the specific embodiments without departing from the spirit or scope of the present disclosure as broadly described. The present embodiments are, therefore, to be considered in all respects as illustrative and not restrictive.

It is to be noted that, throughout the description and claims of this disclosure, the word “comprise” and variations of the word, such as “comprising” and “comprises,” is not intended to exclude other variants or additional components, integers or steps. Modifications and improvements to the invention will be readily apparent to those skilled in the art. Such modifications and improvements are intended to be within the scope of this disclosure.

1.-85. (canceled)

86. A delivery device for deploying an insertable into an orifice guided by digit, the delivery device comprising:
an insertable orientation means having a first longitudinal axis and a base portion, the insertable orientation means adapted to load an insertable positioned in a deployment orientation, and
a skirt member extending from adjacent the insertable orientation means and having a skirt length and comprising opposing digit contacting and orifice contacting surfaces, wherein the skirt member is adapted to be movable substantially parallel to the first longitudinal axis and along the skirt length, between:
an insertable surrounding configuration, wherein an orifice contacting length of the orifice contacting surface substantially faces the insertable orientation means and any loaded insertable; and
a digit surrounding configuration, wherein the orifice contacting length of the orifice contacting surface substantially faces away from the insertable orientation means and any loaded insertable.

87. The delivery device of claim 86 wherein a first portion of the skirt member is adapted to form a digit orientation means as the skirt member is moved along the skirt length from the insertable surrounding configuration toward the digit surrounding configuration, the digit orientation means being adapted to orient at least a portion of at least one digit in a navigation orientation adapted to guide deployment of the insertable into the orifice.

88. The delivery device of claim 86 wherein movement of the skirt member along the skirt length from the insertable surrounding configuration toward the digit surrounding configuration commensurately causes a digit contacting length of the digit contacting surface to face, or to come into contact with, at least a portion of at least one digit when the digit is being surrounded by the skirt member.

89. The delivery device of claim 86 wherein movement of the skirt member along the skirt length from the insertable surrounding configuration toward the digit surrounding configuration commensurately causes a first portion of the orifice contacting length of the orifice contacting surface to face, or to come into contact with, a second portion of the orifice contacting length of the orifice contacting surface.

90. The delivery device of claim 86 wherein following deployment of the insertable into the orifice, the skirt member can be moved from the digit surrounding configuration toward, but short of, the insertable surrounding configuration, adopting a disposable configuration; and wherein at least some of any inter-orifice content withdrawn as the delivery device is withdrawn from the orifice may be generally contained between overlapping portions of the orifice contacting surface, located on the orifice contacting surface facing the insertable orientation means, and/or located in and around the insertable orientation means, when the delivery device is in the disposable configuration.

91. The delivery device of claim 86 wherein the delivery device further comprises at least one lubrication means along, in association with, or applied to, at least a portion of the skirt length, the lubrication means adapted to facilitate movement of the skirt member along the skirt length between the insertable surrounding configuration and the digit surrounding configuration.

92. The delivery device of claim 91 wherein at least one lubrication means is an impression formed in a wall of the skirt member and the impression comprises a series of parallel ridges adapted to enable the movement of the skirt member along the skirt length to be achieved by a substantially rotational movement of an end of the skirt member in a spiral direction along the parallel ridges.

93. The delivery device of claim 91 wherein at least one lubrication means is a kink formed in the wall of the skirt member and the kink is a substantially S-shape in longitudinal section creating a concave and convex inflection point, the kink adapted to enable the movement of the skirt member across the concave and convex inflection points along the skirt length.

94. The delivery device of claim 91 wherein at least one lubrication means comprises a raised, depressed, or raised and depressed pattern formed on the wall of the skirt member, the pattern is formed on an angle essentially preventing inter-engagement of the rises and depressions on a corresponding cross angle pattern when portions of the skirt length are overlapping.

95. The delivery device of claim 91 wherein at least one lubrication means comprises a raised, depressed, or raised and depressed pattern formed on the wall of the skirt member, and the pattern is irregular.

96. The delivery device of claim 94 wherein the pattern is selected from the group consisting of: a crosshatched pattern, a spiral pattern, a honeycomb pattern, a pattern comprising a plurality of approximately half sphere-shaped indents in parallel or in a hexagonal layout, and a corrugated pattern.

97. The delivery device of claim 86 further comprising an insertable ejection means adapted to facilitate ejection of the insertable from the insertable orientation means.

98. The delivery device of claim 97 wherein the insertable ejection means is positioned so as to be adjacent a distal portion of at least one digit in the digit orientation means.

99. The delivery device of claim 97 wherein the insertable ejection means is positioned on an underside of the insertable orientation means base portion.

100. The delivery device of claim 97 wherein the insertable ejection means is a pressable member that can be pressed by a distal portion of at least one digit in the digit orientation means, so as to cause a proximal end of the insertable to be ejected from the insertable orientation means.

101. The delivery device of claim 97 wherein a portion of the digit orientation means adjacent the insertable ejection means can accommodate a distal length of a fingernail.

102. The delivery device of claim 86 wherein the skirt member further comprises an insertable component receiving means adapted to receive a component of the insertable; and wherein the insertable component receiving means is selected from the group consisting of: a skirt member width channel interposed between an opening on the digit contacting surface and an opening on the orifice contacting surface, and a skirt member width slit-type channel opening at a distal end of the skirt member and extending along at least a portion of the skirt length.

103. The delivery device of claim 86 wherein a distal end of the skirt member is thickened relative to a remainder of the skirt member.

104. The delivery device of claim 103 wherein the thickened end portion is adapted to facilitate movement of the skirt member along the skirt length between the insertable surrounding configuration and the digit surrounding configuration.

105. The delivery device of claim 86, wherein the digit orientation means and the skirt member are integrally formed.
